(19) United States
(12) Patent Application Publication
(10) Pub. No.: US 2010/0087475 A1
(43) Pub. Date: Apr. 8, 2010

(54) PHARMACEUTICAL COMPOUNDS

(75) Inventors: Andrew John Duffield, Berkhamsted (GB); Grant Johnston, Northampton (GB); Brendan Mark Green, Northampton (GB); John Christopher Townsley, Northampton (GB)

Correspondence Address: SCHWEGMAN, LUNDBERG & WOESSNER, P.A., P.O. BOX 2938, MINNEAPOLIS, MN, 55402, US

(73) Assignee: Biovali Laboratories International (Barbados) S.R.L., Christ Church (BB)

(21) Appl. No.: 12/555,720

(22) Filed: Sep. 8, 2009

(30) Foreign Application Priority Data
Sep. 8, 2008 (GB) 0816372.7

(51) Int. Cl. A61K 31/4375; C07D 455/06; A61P 3/10; A61P 11/06; A61P 25/00

(52) U.S. Cl. 514/294; 546/95

(57) ABSTRACT

This application describes substituted 1,3,4,6,7,11b-hexahydro-benzo(a)quinolizine compounds, pharmaceutical compositions containing them, processes for making them and their therapeutic methods.

### RELATED APPLICATIONS

This application claims benefit of the filing date of UK Patent Application No. 0816372.7 filed Sep. 8, 2008, the contents of which are specifically incorporated herein in their entirety.

### FIELD OF THE INVENTION

This invention relates to substituted 1,3,4,6,7,11b-hexahydro-benzo(a)quinolizine compounds, pharmaceutical compositions containing them, processes for making them and their therapeutic methods.

### BACKGROUND OF THE INVENTION

Tetrabenazine (Chemical name: 1,3,4,6,7,11b-hexahydro-9,10-dimethoxy-3-(2-methylpropyl)-2H-benzo(a)quinolizin-2-one) has been in use as a pharmaceutical drug since the late 1950s. Initially used as an anti-psychotic, tetrabenazine is currently used for treating hyperkinetic movement disorders such as Huntington's disease, hemiballismus, senile chorea, tic, tardive dyskinesia, dystonia, myoclonus and Tourette's syndrome, see for example Ondo et al., Am. J. Psychiatry. (1999) August; 156(8):1279-81 and Jankovic et al., Neurology (1997) February; 48(2):358-62. The primary pharmacological action of tetrabenazine is to reduce the supply of monoamines (e.g. dopamine, serotonin, and norepinephrine) in the central nervous system by inhibiting the human vesicular monoamine transporter isoform 2 (hVMAT2). The drug also blocks postsynaptic dopamine receptors. Tetrabenazine is an effective and safe drug for the treatment of a variety of hyperkinetic movement disorders and, in contrast to typical neuroleptics, has not been demonstrated to cause tardive dyskinesia.

Nevertheless, tetrabenazine does exhibit a number of dose-related side effects including depression, parkinsonism, drowsiness, nervousness or anxiety, insomnia and, in rare cases, neuroleptic malignant syndrome. The central effects of tetrabenazine closely resemble those of reserpine, but it differs from reserpine in that it lacks activity at the VMAT1 transporter. The lack of activity at the VMAT1 transporter means that tetrabenazine has less peripheral activity than reserpine and consequently does not produce VMAT1-related side effects such as hypotension.

### SUMMARY OF THE INVENTION

The present invention relates to des-methyl derivatives of tetrabenazine and dihydrotetrabenazine, compositions and methods of using these novel des-methyl derivatives.

One aspect of the invention is a compound of the formula (1):

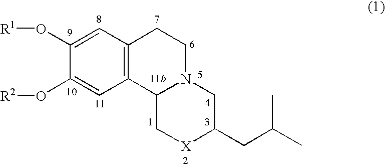

wherein R¹ and R² are each selected from hydrogen and methyl; and
X is CHOH or C═O; or
a pharmaceutically acceptable salt or tautomer thereof.

In some embodiments, the compounds of formula (1) exclude compounds 2R,3R,11bR-9-O-desmethyl-dihydrotetrabenazine and 2S,3S,11bS-9-O-desmethyl-dihydrotetrabenazine.

In some embodiments, the compound of formula (1) has formula (1a):

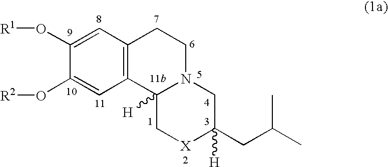

or a pharmaceutically acceptable salt or tautomer thereof, wherein R¹ and R² are each selected from hydrogen and methyl and X is CHOH or C═O. In some embodiments, the compounds of formula (1a) have at least one hydrogen for R¹ or R². Also in some embodiments, the hydrogen atoms in positions 3 and 11b are present in a cis relative orientation when (i) R¹ and R² are both hydrogen, or (ii) R¹ is hydrogen and R² is methyl.

One sub-group of compounds of the formula (1a) is constituted by compounds in which the hydrogen atoms at the 3- and 11b-positions are in the cis relative orientation.

An example of a compound of formula (1) is compound having the formula (2):

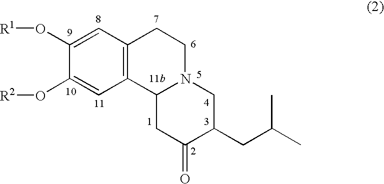

and pharmaceutically acceptable salts thereof.

An example of a compound of formula (2) is a compound having formula (2a):

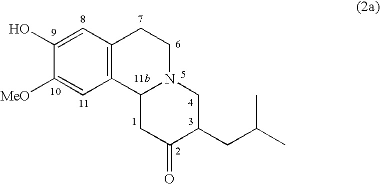

and pharmaceutically acceptable salts thereof.

Examples of compounds of formula (2a) include compounds such as compounds (2a-I) and (2a-II):

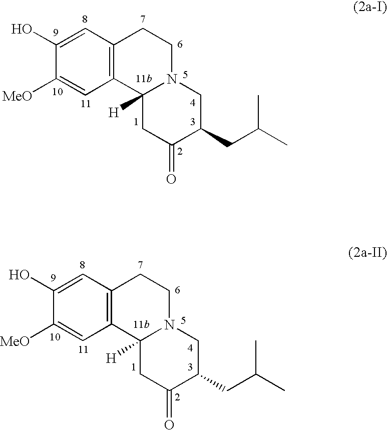

and pharmaceutically acceptable salts thereof.

Another example of a compound of formula (2) is compound having formula (2b):

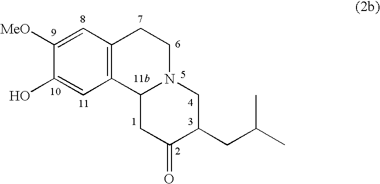

and pharmaceutically acceptable salts thereof.

Examples of compounds of formula (2b) include compounds (2b-I) and (2b-II):

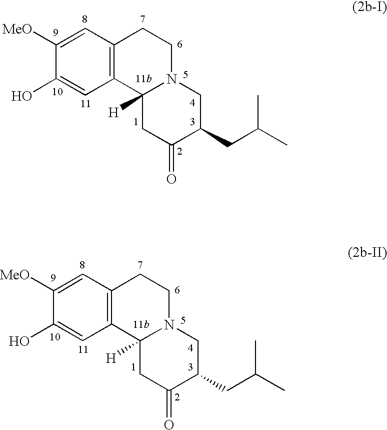

and pharmaceutically acceptable salts thereof.

Another example of a compound of formula (2) is a compound having formula (2c):

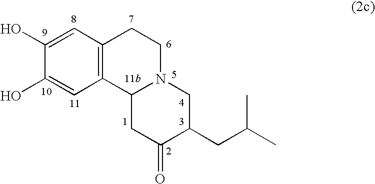

and pharmaceutically acceptable salts thereof.

Examples of compounds of formula (2c) include compounds (2c-I) and (2c-II):

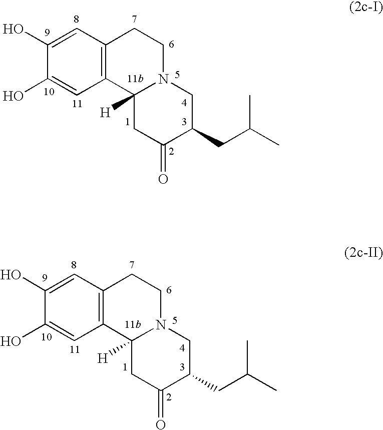

and pharmaceutically acceptable salts thereof.

Another example of a compound of formula (1) is a compound having formula (3):

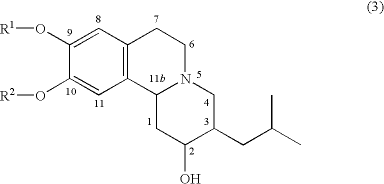

and pharmaceutically acceptable salts thereof, but excluding compounds 2R,3R,11bR-9-O-desmethyl-dihydrotetrabenazine and 2S,3S,11bS-9-O-desmethyl-dihydrotetrabenazine and their salts and tautomers.

One example of a compound of formula (3) is compound having formula (3a):

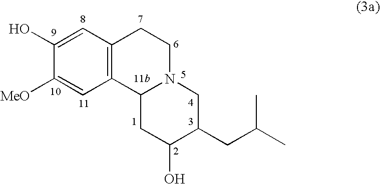

and pharmaceutically acceptable salts thereof.

Examples of compounds of formula (3a) include compounds (3a-I) and (3a-II):

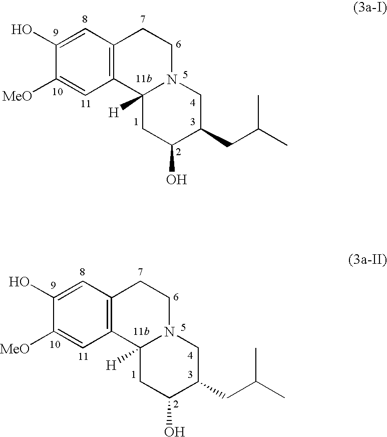

and salts and tautomers thereof.

Other examples of a compound of formula (3a) are compounds (3a-V), (3a-VI), (3a-VII) and (3a-VIII):

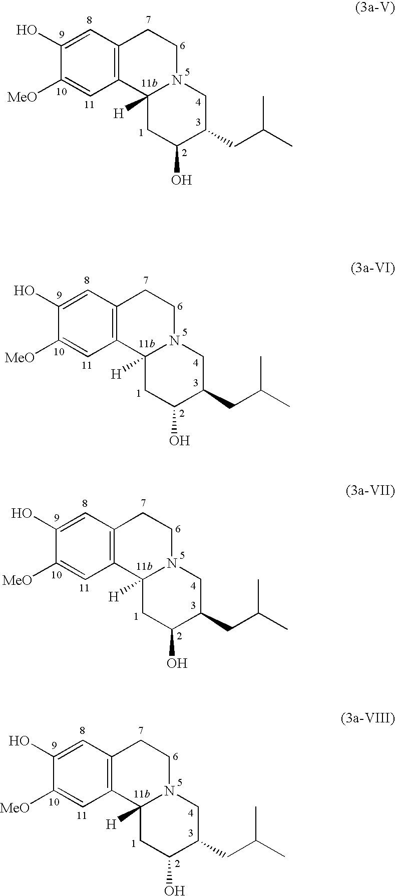

and pharmaceutically acceptable salts thereof.

Another example of a compound of formula (3) is compound having formula (3b):

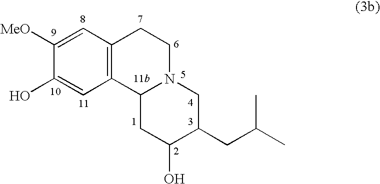

and pharmaceutically acceptable salts thereof.

Examples of a compound of formula (3b) include compounds (3b-I), (3b-II), (3b-III) and (3b-IV):

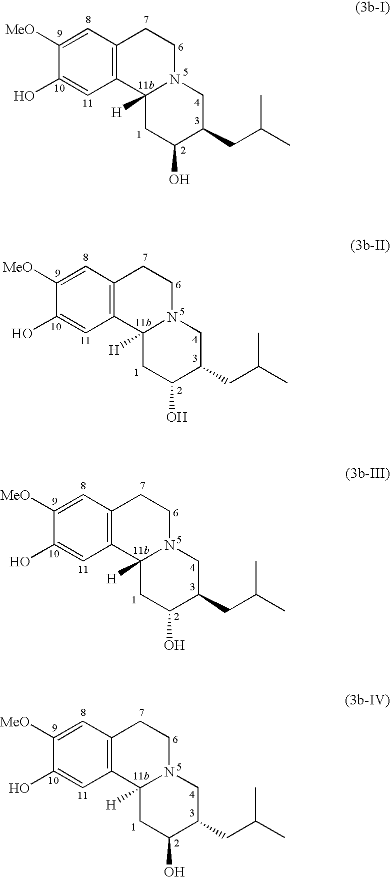

and pharmaceutically acceptable salts thereof.

Another example of a compound of formula (3b) is one of compounds (3b-V), (3b-VI) (3b-VII) and (3b-VIII):

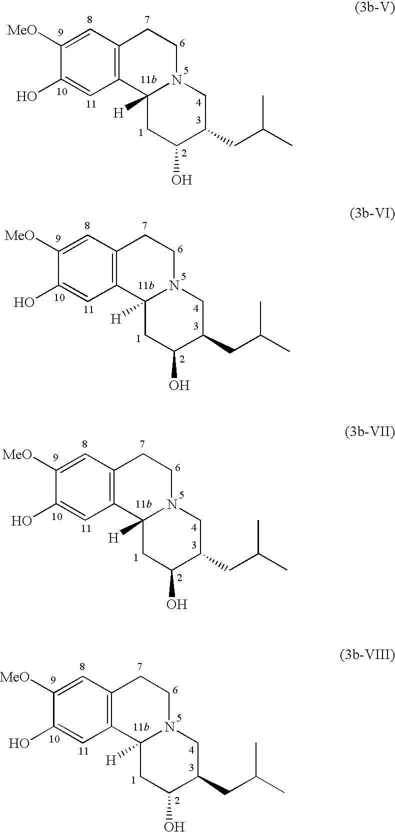

and pharmaceutically acceptable salts thereof.

Another example of a compound of formula (3) is a compound having formula (3c):

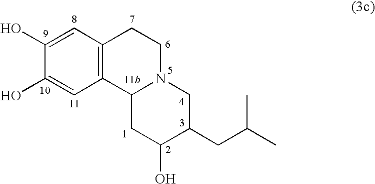

and pharmaceutically acceptable salts thereof.

Examples of a compound of formula (3c) are compounds (3c-I), (3c-II), (3c-III) and (3c-IV):

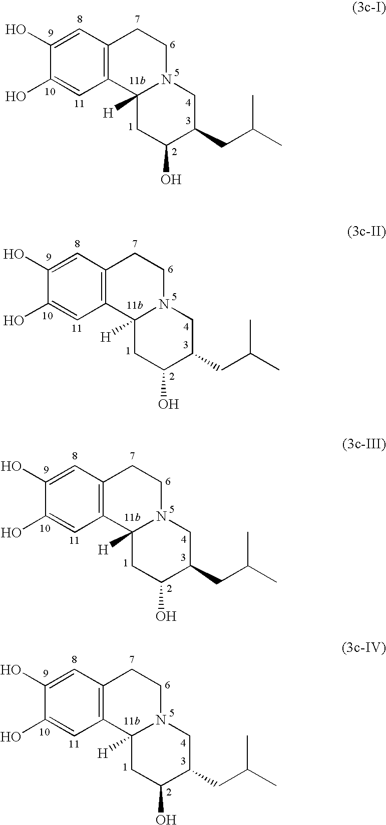

and pharmaceutically acceptable salts thereof.

Other examples of a compound of formula (3c) include compounds (3c-V), (3c-VI) (3c-VII) and (3c-VIII):

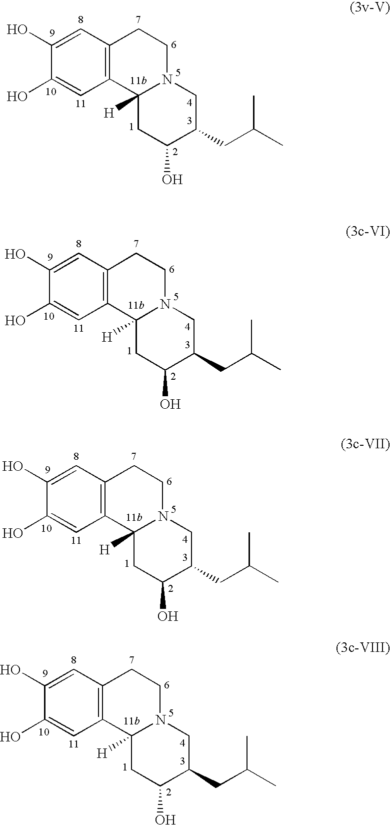

and pharmaceutically acceptable salts thereof.

Another aspect of the invention is a pharmaceutical composition that includes any of the compounds described herein, for example, any compound of the formula (1):

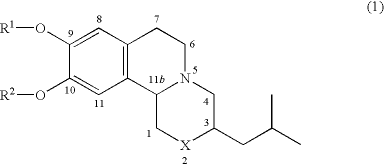

or a pharmaceutically acceptable salt or tautomer thereof;
wherein R¹ and R² are each selected from hydrogen and methyl; and
X is CHOH or C═O;
but excluding compounds 2R,3R,11bR-9-O-desmethyl-dihydrotetrabenazine and 2S,3S,11bS-9-O-desmethyl-dihydrotetrabenazine and their salts and tautomers;
and a pharmaceutically acceptable carrier.

Such compositions can include a therapeutically effective amount of any of the compounds described herein (e.g., any of the compounds of formula (1)).

Another aspect of the invention is a method of treating a movement disorder in a patient in need thereof comprising administrating to the patient any of the compositions containing one or more of the compounds described herein to thereby treat the movement disorder in the patient. For example, the movement disorder can be Huntington's disease, or Tourette's syndrome. Such a method can also halt or slow the development of Huntington's disease or Tourette's syndrome.

Another aspect of the invention is a method of treating an inflammatory disease in a patient in need thereof comprising administrating to the patient any of the compositions containing one or more of the compounds described herein to thereby treat the inflammatory disease in the patient.

Another aspect of the invention is a method of treating multiple sclerosis in a patient in need thereof comprising administrating to the patient any of the compositions containing one or more of the compounds described herein to thereby treat the multiple sclerosis in the patient.

Another aspect of the invention is a method of treating a psychoses in a patient in need thereof comprising administrating to the patient any of the compositions containing one or more of the compounds described herein to thereby treat the psychoses in the patient. For example, the psychoses can be schizophrenia. The method can also treat a cognitive deficit associated with schizophrenia.

Another aspect of the invention is a method of treating anxiety in a patient in need thereof comprising administrating to the patient any of the compositions containing one or more of the compounds described herein to thereby treat the anxiety in the patient.

Another aspect of the invention is a method of treating depression in a patient in need thereof comprising administrating to the patient any of the compositions containing one or more of the compounds described herein to thereby treat the depression in the patient.

Another aspect of the invention is a method of treating a cognitive deficit in a patient in need thereof comprising administrating to the patient any of the compositions containing one or more of the compounds described herein to thereby treat the cognitive deficit in the patient.

Another aspect of the invention is a method of treating dementia in a patient in need thereof comprising administrating to the patient any of the compositions containing one or more of the compounds described herein to thereby treat the dementia in the patient.

Another aspect of the invention is a method of treating asthma in a patient in need thereof comprising administrating to the patient any of the compositions containing one or more of the compounds described herein to thereby treat the asthma in the patient.

Another aspect of the invention is a method of treating diabetes mellitus in a patient in need thereof comprising administrating to the patient any of the compositions containing one or more of the compounds described herein to thereby treat the diabetes mellitus in the patient.

### DETAILED DESCRIPTION OF THE INVENTION

The invention generally relates to novel substituted 1,3,4,6,7,11b-hexahydro-benzo(a)quinolizine compounds, pharmaceutical compositions containing them, processes for making them and their therapeutic methods.

Compounds described herein have some of the desirable properties of tetrabenazine but are structurally distinct from tetrabenazine. The compounds described herein have excellent activity and may also avoid some of the side effects of tetrabenazine. The chemical structure of tetrabenazine is shown below.

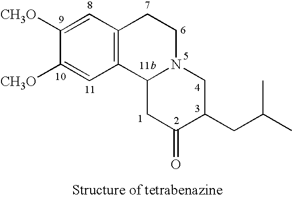

Tetrabenazine has chiral centres at the 3 and 11b carbon atoms and hence can, theoretically, exist in a total of four isomeric forms, as shown below.

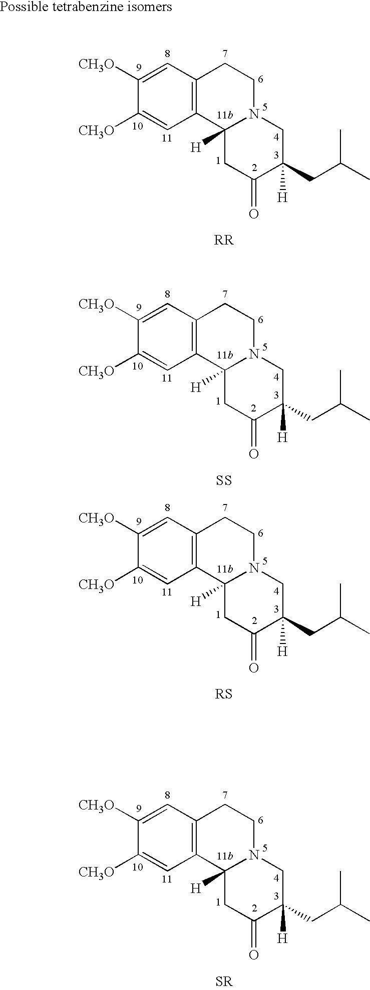

The stereochemistry of each tetrabenazine isomer is defined using the “R and S” nomenclature developed by Cahn, Ingold and Prelog, see Advanced Organic Chemistry by Jerry March, 4^{th} Edition, John Wiley & Sons, New York, 1992, pages 109-114. As used herein, the designations “R” or “S” are given in the order of the position numbers of the carbon atoms. Thus, for example, RS is a shorthand notation for 3R,11bS. Similarly, when three chiral centres are present, as in the dihydrotetrabenazines described below, the designations “R” or “S” are listed in the order of the carbon atoms 2, 3 and 11b. Thus, the 2S,3R,11bR isomer is referred to in short hand form as SRR, and so on.

Commercially available tetrabenazine is a racemic mixture of the RR and SS isomers and available evidence indicates that the RR and SS isomers are the most thermodynamically stable isomers. The RR and SS isomers are referred to individually or collectively herein as trans-tetrabenazine because the hydrogen atoms at the 3 and 11b positions have a trans relative orientation.

In this application, conventional notation is used to indicate the stereochemical orientation of a bond. Thus, unless the context indicates otherwise, a wedge-shaped bond [symbol] in a structural drawing indicates that the bond extends upwards relative to the plane of the page whereas a dotted line bond [symbol] indicates that the bond extends downwards relative to the plane of the page. A straight line of uniform thickness ( [symbol] )does not carry any stereochemical implications (unless the context indicates otherwise) whereas a wavy line [symbol] indicates that a bond can be either up or down relative to the plane of the page.

Tetrabenazine has somewhat poor and variable bioavailability. It is extensively metabolised by first-pass metabolism, and little or no unchanged tetrabenazine is typically detected in the urine. The major metabolite is dihydrotetrabenazine (Chemical name: 2-hydroxy-3-(2-methylpropyl)-1,3,4,6,7,11b-hexahydro-9,10-dimethoxy-benzo(a)quinolizine) which is formed by reduction of the 2-keto group in tetrabenazine, and is believed to be primarily responsible for the activity of the drug (see Mehvar et al., Drug Metab. Disp, 15, 250-255 (1987) and J. Pharm. Sci., 76, No. 6, 461-465 (1987)).

There are eight dihydrotetrabenazine isomers. Four of the isomers are derived from the more stable RR and SS isomers of the parent tetrabenazine and have a trans relative orientation between the hydrogen atoms at the 3 and 11 b positions. The structures of the four 3,11b trans dihydrotetrabenazine isomers are shown below.

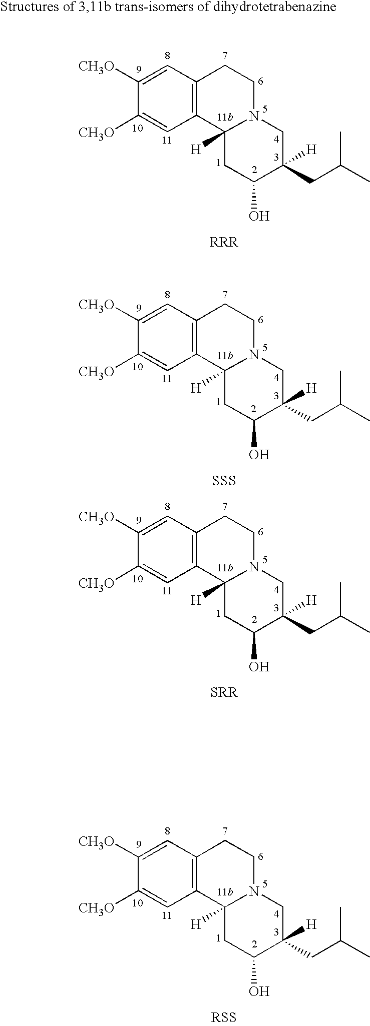

The four 3,11b cis-isomers are disclosed in earlier patent applications filed by applicants, including WO2005/077946, WO2007/007105, WO2007/017643 and WO2007/017654, which are specifically incorporated herein in their entireties. The structures of the 3,11b cis isomers are as follows:

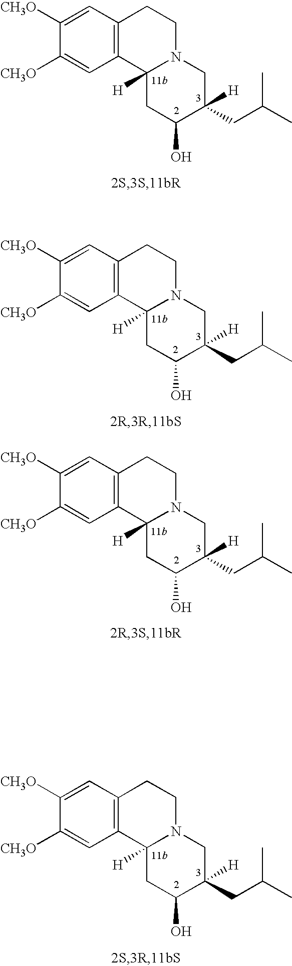

The above 3,11b cis-dihydrotetrabenazine isomers are disclosed as having a number of therapeutic uses including use in treating movement disorders (WO2005/077946), use in arresting or slowing the development of the symptoms of Huntington's disease (WO2007/007105), anti-inflammatory use (WO2007/017643), and anti-psychotic use (WO2007/017654). See also, Kilbourn et al., Chirality, 9:59-62 (1997) providing description of compounds prepared as substrates for X-ray crystallographic studies for the purpose of determining the absolute structures of (+) α-dihydrotetrabenazine, including 9-desmethyl analogues of the RRR and SSS dihydrotetrabenazines.

One aspect of the invention is a compound of formula (1):

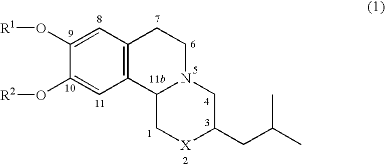

or a pharmaceutically acceptable salt or tautomer thereof;
wherein R¹ and R² are each selected from hydrogen and methyl; and

### X is CHOH or C═O;

and a pharmaceutically acceptable carrier.

In some embodiments, the compounds of the formula (1) have at least one of R¹ and R² as hydrogen. In other embodiments, the compound of the formula (1) excludes compounds 2R,3R,11bR-9-O-desmethyl-dihydrotetrabenazine and 2S,3S,11bS-9-O-desmethyl-dihydrotetrabenazine.

Also provided herein are compounds of formula (1a):

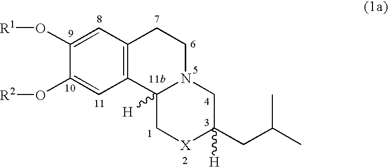

or a pharmaceutically acceptable salt or tautomer thereof, wherein R¹ and R² are each selected from hydrogen and methyl, wherein at least one of R¹ and R² is hydrogen; and X is CHOH or C═O. In some embodiments, the hydrogen atoms in positions 3 and 11b are present in a cis relative orientation when (i) R¹ and R² are both hydrogen, or (ii) when R¹ is hydrogen and R² is methyl.

One sub-group of compounds of the formula (1a) is constituted by compounds in which the hydrogen atoms at the 3- and 11b-positions are in the cis relative orientation.

Within formula (1a), one group of compounds can be represented by the formula (2):

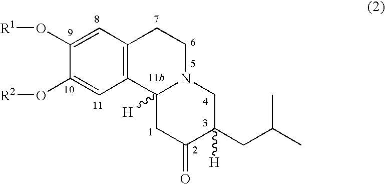

and pharmaceutically acceptable salts thereof.

One sub-group of compounds within formula (2) is represented by formula (2a):

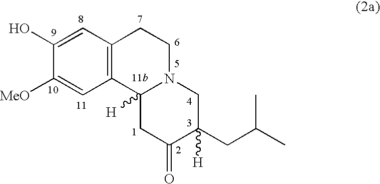

and pharmaceutically acceptable salts thereof.

Within formula (2a), particular compounds are the compounds (2a-I) and (2a-II):

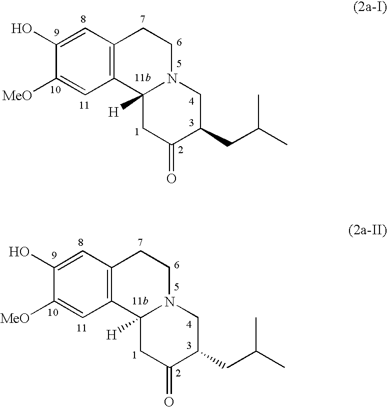

and pharmaceutically acceptable salts thereof. In some embodiments, the invention excludes any of compounds (2a-I) and/or (2a-II).

Another particular sub-group of compounds within formula (2) is represented by formula (2b):

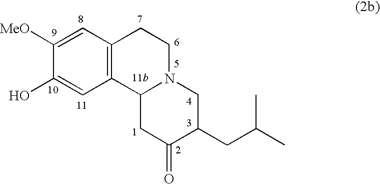

and pharmaceutically acceptable salts thereof.

Within formula (2b), particular compounds are the compounds (2b-I) and (2b-II):

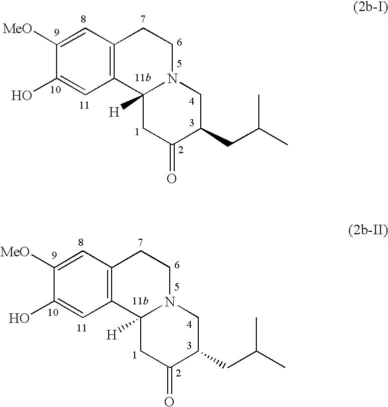

and pharmaceutically acceptable salts thereof.

Another particular sub-group of compounds within formula (2) is represented by formula (2c):

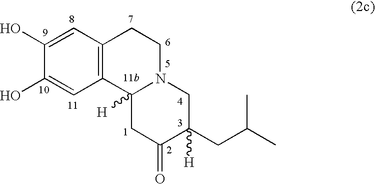

and pharmaceutically acceptable salts thereof.

Examples of compounds of formula (2c) include compounds (2c-I) and (2c-II):

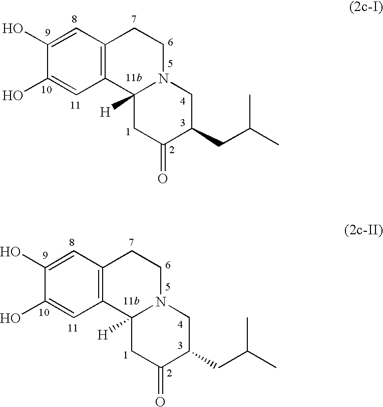

and pharmaceutically acceptable salts thereof. In some embodiments, the invention excludes any of compounds (2c-I) and/or (2c-II).

Another group of compounds within formula (1) is represented by formula (3):

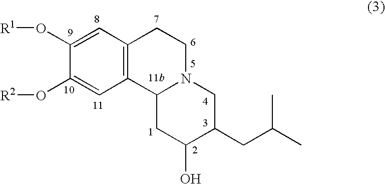

and pharmaceutically acceptable salts thereof.

One particular sub-group of compounds within formula (3) is represented by formula (3a):

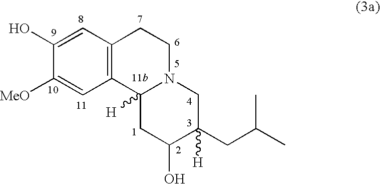

and pharmaceutically acceptable salts thereof.

Examples of compounds of formula (3a) include compounds (3a-I), (3a-II), (3a-III), (3-a-IV), (3a-V), (3a-VI), (3a-VII) and (3a-VIII):

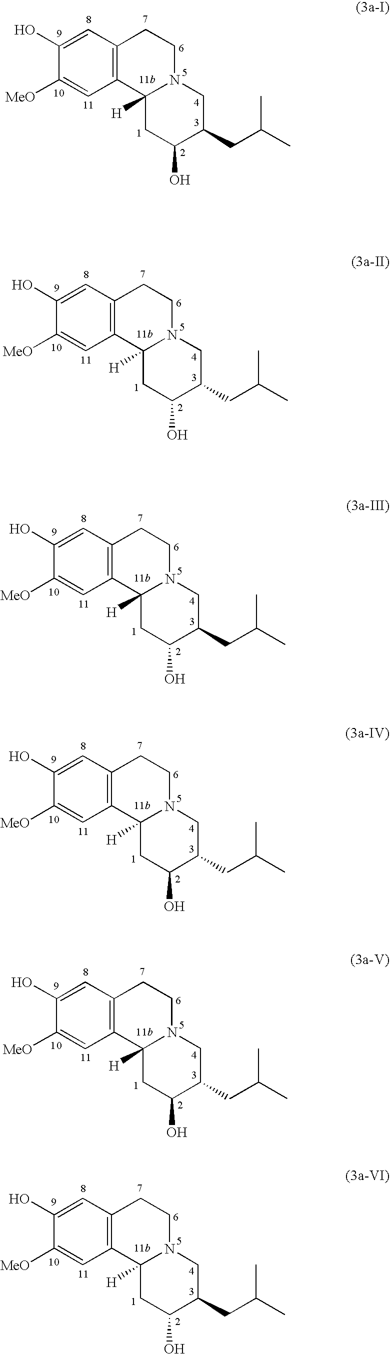

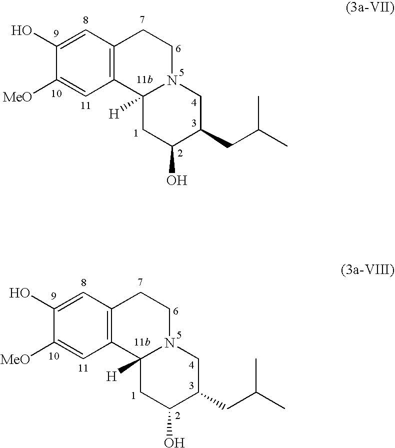

and pharmaceutically acceptable salts thereof. In some embodiments, the invention excludes any of compounds (3a-I), (3a-II), (3a-III), and/or (3a-IV).

Compounds (3a-III) and (3a-IV) may previously have been disclosed only as substrates for X-ray crystallographic studies (see Kilbourn et al., Chirality, 9:59-62 (1997); see, also DaSilva et al., Nuclear Medicine and Biology, Vol. 21, No. 2, pages 151-156, (1994)). Accordingly, in some embodiments, these compounds do not per se form part of the group of compounds of the invention. For example, within formula (3a), a sub-group of compounds constituted by compounds having a 3,11b-trans-stereochemical configuration, e.g. compounds (3a-I) and (3a-II) may be excluded and not be part of the invention.

Another sub-group of compounds is constituted by compounds having a 3,11b-cis stereochemical configuration, i.e. compounds (3a-V), (3a-VI), (3a-VII) and (3a-VIII).

Another particular sub-group of compounds within formula (3) is represented by formula (3b):

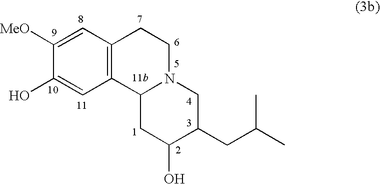

and pharmaceutically acceptable salts thereof.

Examples of compounds of formula (3b) include compounds (3b-I), (3b-II), (3b-III), (3b-IV) (3b-V), (3b-VI) (3b-VII) and (3b-VIII):

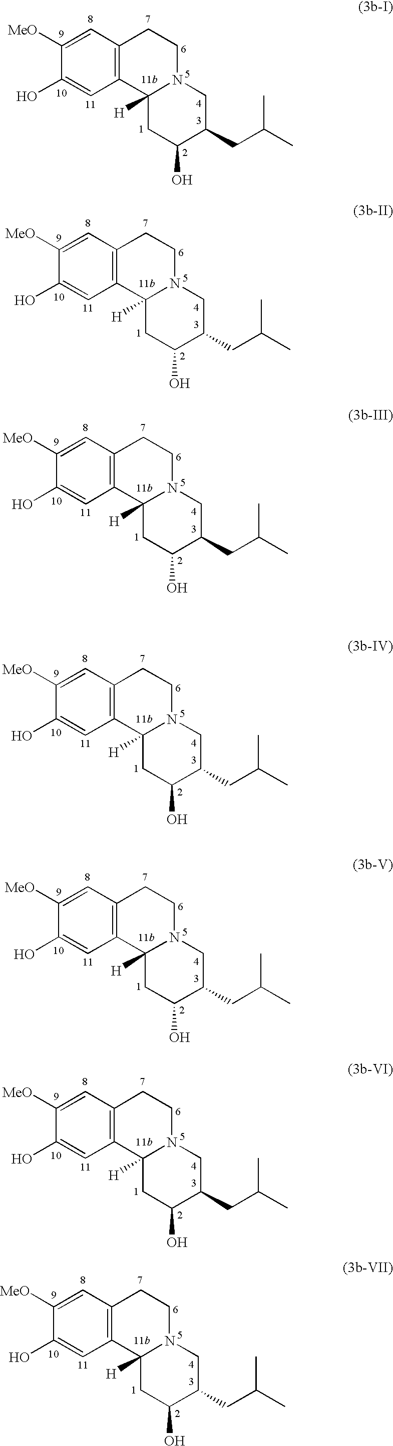

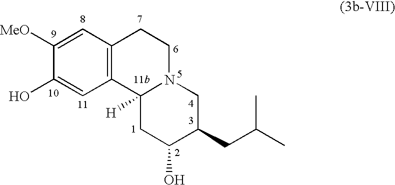

and pharmaceutically acceptable salts thereof.

Within formula (3b), one sub-group of compounds is constituted by compounds having a 3,11b-trans-stereochemical configuration, i.e. compounds (3b-I), (3b-II), (3b-III) and (3b-IV).

Another sub-group of compounds is constituted by compounds having a 3,11b-cis stereochemical configuration, i.e. compounds (3b-V), (3b-VI), (3b-VII) and (3b-VIII).

Another particular sub-group of compounds within formula (3) is represented by formula (3c):

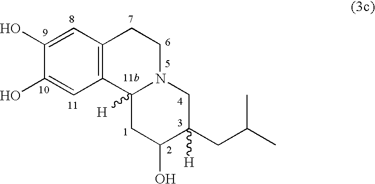

and pharmaceutically acceptable salts thereof.

Within formula (3c), particular compounds are the compounds (3c-I), (3c-II), (3c-III), (3c-IV) (3c-V), (3c-VI) (3c-VII) and (3c-VIII):

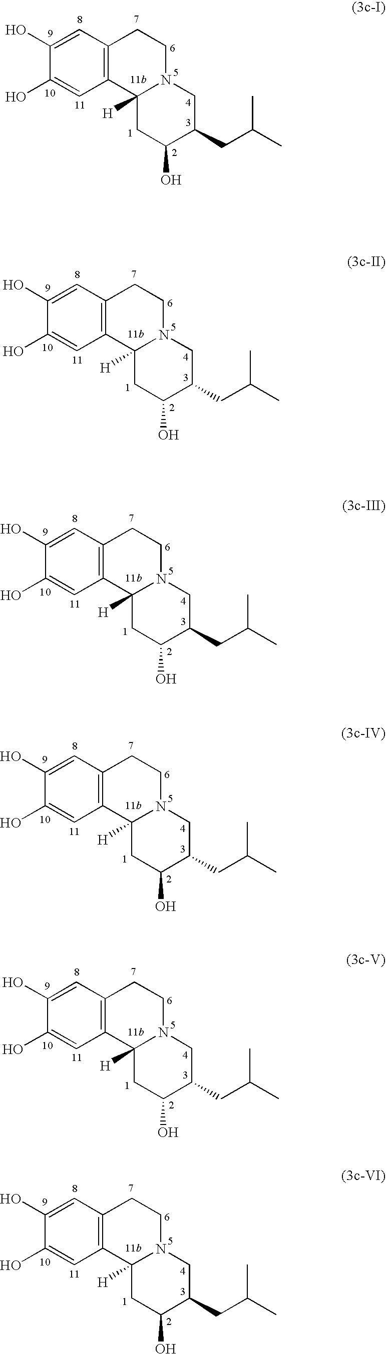

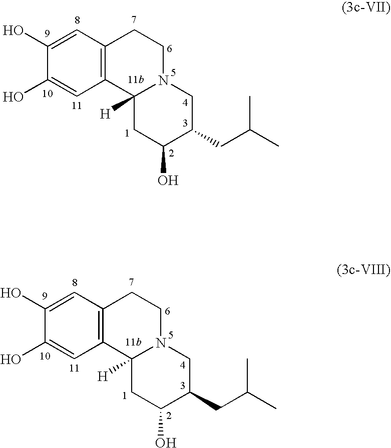

and pharmaceutically acceptable salts thereof. However, in some embodiments any of compounds (3c-I), (3c-II), (3c-III), and/or (3c-IV) may be excluded from the invention. Thus, a sub-group of compounds of formula (3c) which is constituted by compounds having a 3,11b-trans-stereochemical configuration, i.e. compounds (3c-I), (3c-II), (3c-III) and (3c-IV), may be excluded and not be part of the invention.

Another sub-group of compounds can be part of the invention including those constituted by compounds having a 3,11b-cis stereochemical configuration, i.e. compounds (3b-V), (3b-VI), (3b-VII) and (3b-VIII).

All of the compounds described above can be formulated into pharmaceutical compositions and used in the methods of the invention. Although the structures of some compounds may have previously been described, applicants are of the belief that the utility of such compounds and/or their use in pharmaceutical compositions has not yet have been recognized. For example, compounds falling within the scope of formula (1) and which may be formulated and employed in the compositions and methods of the invention are the compounds (2a-I) and (2a-II):

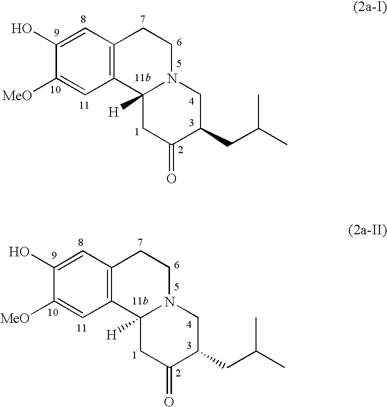

and pharmaceutically acceptable salts thereof; and the compounds (2c-I) and (2c-II):

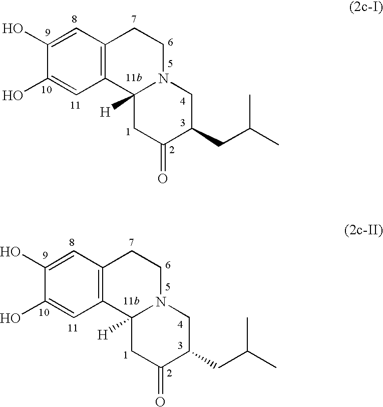

and pharmaceutically acceptable salts thereof; and the compounds (3a-I), (3a-II), (3a-III) and (3-a-IV):

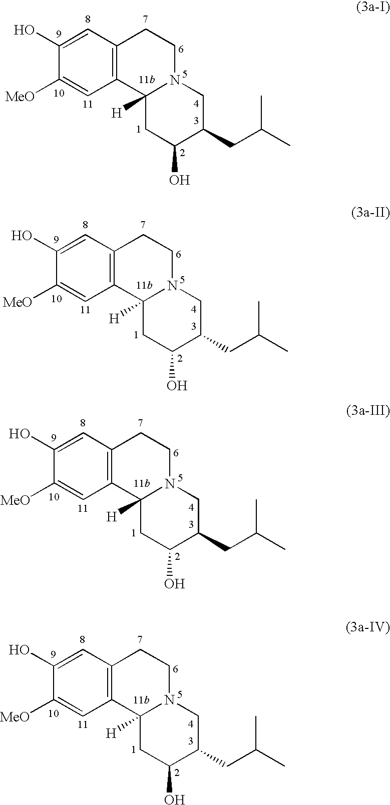

and pharmaceutically acceptable salts thereof; and the compounds (3c-I), (3c-II), (3c-III), (3c-IV):

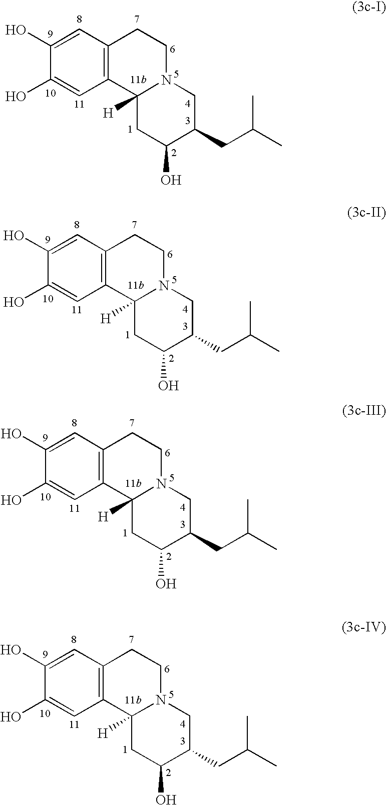

and pharmaceutically acceptable salts thereof.

In further aspects, the invention provides:
Compound (2a-I) per se, or a pharmaceutically acceptable salt thereof.
Compound (2a-II) per se, or a pharmaceutically acceptable salt thereof.
Compound (2c-I) per se, or a pharmaceutically acceptable salt thereof.
Compound (2c-II) per se, or a pharmaceutically acceptable salt thereof.
Compound (3a-I) per se, or a pharmaceutically acceptable salt thereof.
Compound (3a-II) per se, or a pharmaceutically acceptable salt thereof.
Compound (3c-I) per se, or a pharmaceutically acceptable salt thereof.
Compound (3c-II) per se, or a pharmaceutically acceptable salt thereof.
Compound (3c-III)per se, or a pharmaceutically acceptable salt thereof.
Compound (3c-IV) per se, or a pharmaceutically acceptable salt thereof.

In this application, compounds in which the hydrogen atoms at the 3- and 11b-positions are in the trans relative configuration, and the hydrogen atoms at the 2- and 3-positions are also in the trans relative configuration, may be referred to herein by the designation “α”.

Compounds in which the hydrogen atoms at the 3- and 11b-positions are in the trans relative configuration, and the hydrogen atoms at the 2- and 3-positions are also in the cis relative configuration, may be referred to herein by the designation “β”.

Compounds in which the hydrogen atoms at the 3- and 11b-positions are in the cis relative configuration, and the hydrogen atoms at the 2- and 3-positions are in the trans relative configuration, may be referred to herein by the designation “γ”.

In summary, the compound products provided herein can have formula (1a):

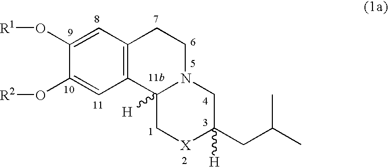

or a pharmaceutically acceptable salt or tautomer thereof, wherein R¹ and R² are each selected from hydrogen and methyl, provided that at least one of R¹ and R² is hydrogen; and X is CHOH or C═O;
and provided that the hydrogen atoms in positions 3 and 11b are present in a cis relative orientation when (i) R¹ and R² are both hydrogen, or (ii) R¹ is hydrogen and R² is methyl.

Compounds of formula (1a) (and formula (1)) can have the hydrogen atoms in positions 3 and 11b are present in a cis relative orientation.

One group of formula (1a) compounds have formula (2):

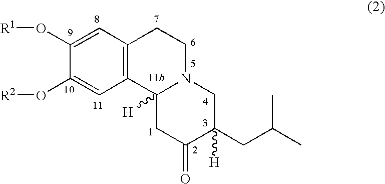

and pharmaceutically acceptable salts thereof.

One subgroup of formula (2) compounds have formula (2a):

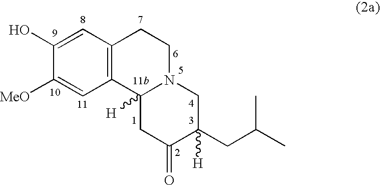

and pharmaceutically acceptable salts thereof.

Another subgroup of formula (2) compounds have formula (2b):

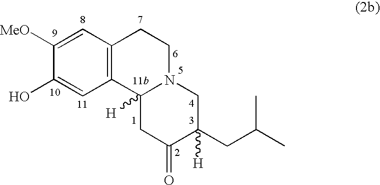

and pharmaceutically acceptable salts thereof.

Examples of compounds of formula (2b) include compounds (2b-I) and (2b-II):

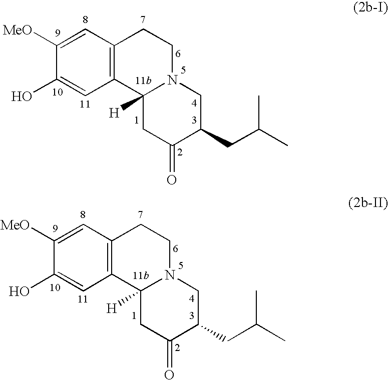

and pharmaceutically acceptable salts thereof.

Another subgroup of compounds have formula (2c):

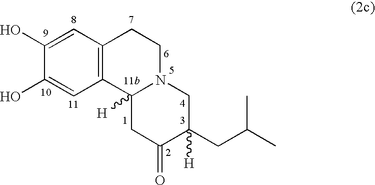

and pharmaceutically acceptable salts thereof.

Another group of formula (1a) compounds have the formula (3):

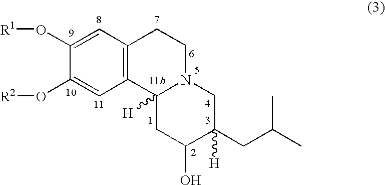

and pharmaceutically acceptable salts thereof.

One subgroup of formula (3) compounds have formula (3a):

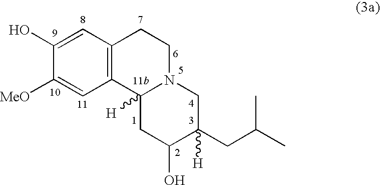

and pharmaceutically acceptable salts thereof.

Other example of compounds of compounds of formula (3a) include compounds (3a-V), (3a-VI), (3a-VII) and (3a-VIII):

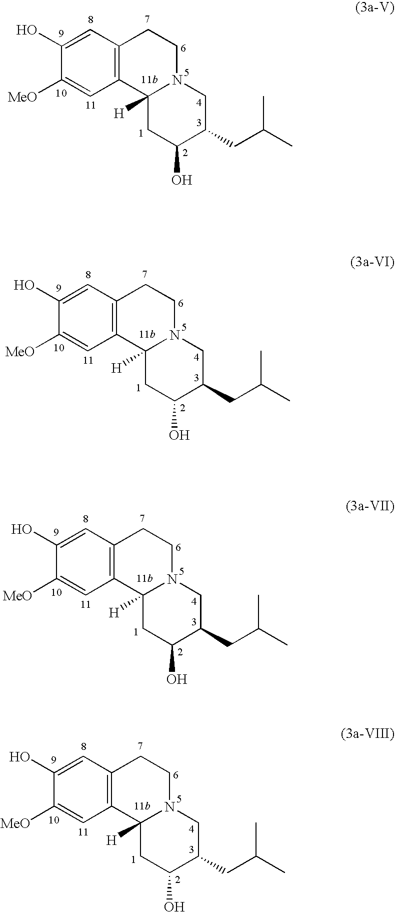

and pharmaceutically acceptable salts thereof.

Another subgroup of compounds have formula (3b):

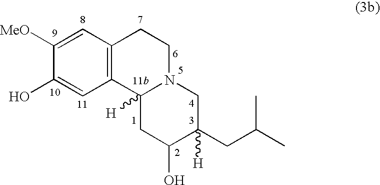

and pharmaceutically acceptable salts thereof.

Examples of compounds if formula (3b) include compounds (3b-I), (3b-II), (3b-III) and (3b-IV):

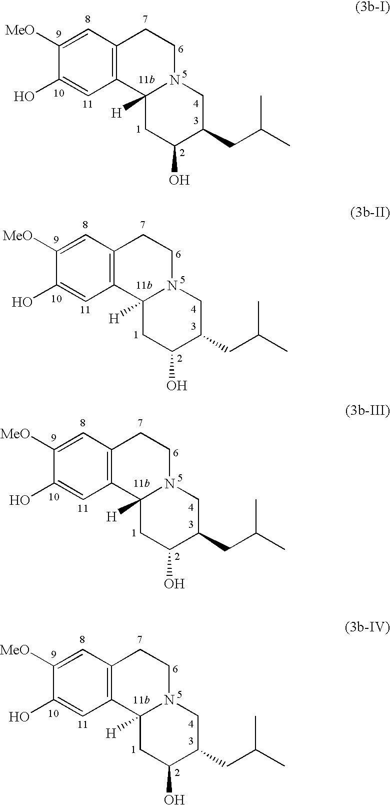

and pharmaceutically acceptable salts thereof.

Other examples of formula (3b) compounds include compounds (3b-V), (3b-VI) (3b-VII) and (3b-VIII):

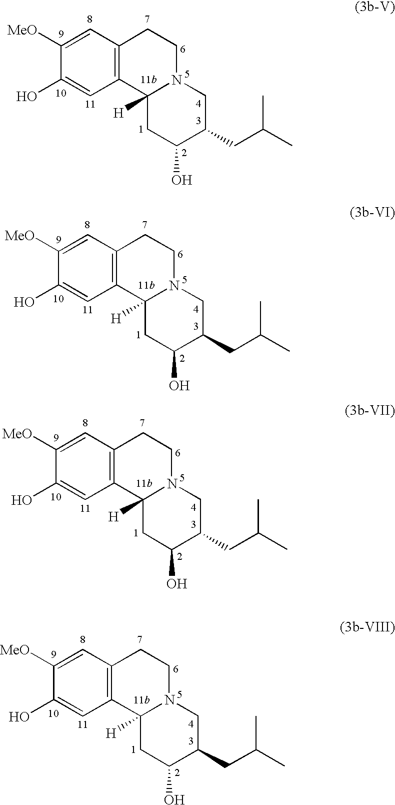

and pharmaceutically acceptable salts thereof.

Another subgroup of compounds of the invention have formula (3c):

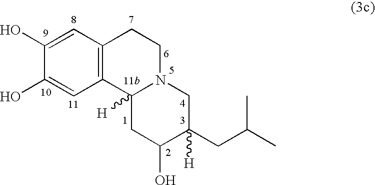

and pharmaceutically acceptable salts thereof.

The compound can also be selected from the compounds (3c-V), (3c-VI) (3c-VII) and (3c-VIII):

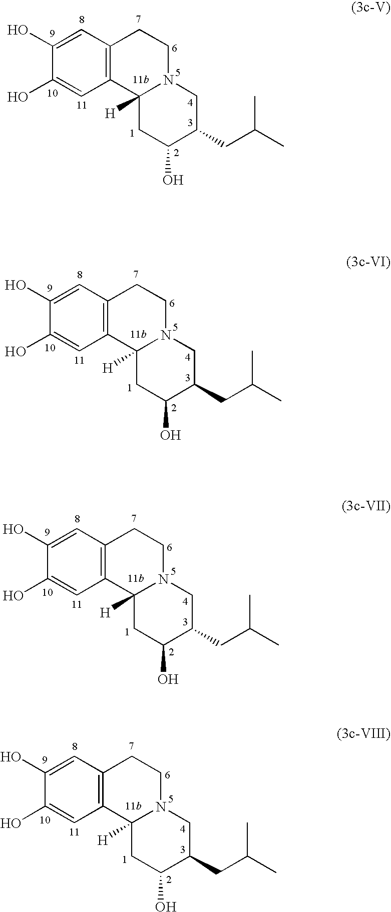

and pharmaceutically acceptable salts thereof.

One example of a compound of the invention is (−)-10-desmethyl-γ-dihydrotetrabenazine or a pharmaceutically acceptable salt thereof. Another example of a compound of the invention is (+)-10-desmethyl-γ-dihydrotetrabenazine or a pharmaceutically acceptable salt thereof.

Compounds of the formula (1) can, in some embodiments, include compounds selected from the compounds (2a-I) and (2a-II):

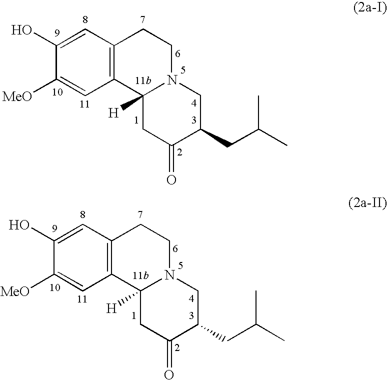

and/or pharmaceutically acceptable salts thereof; and the compounds (2c-I) and (2c-II):

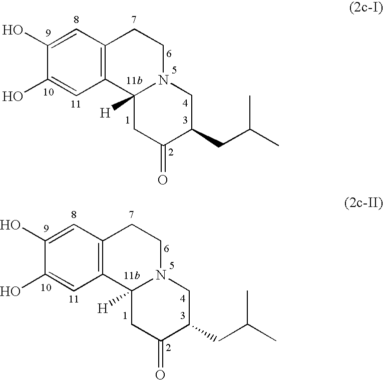

and/or pharmaceutically acceptable salts thereof; and the compounds (3a-I), (3a-II), (3a-III) and (3-a-IV):

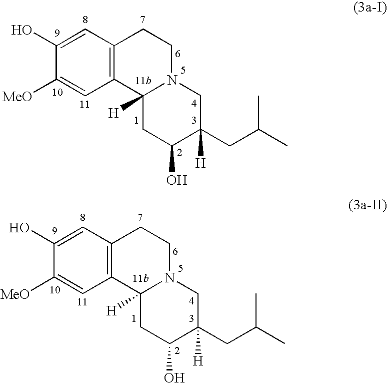

and/or pharmaceutically acceptable salts thereof; and the compounds (3c-I), (3c-II), (3c-III), (3c-IV):

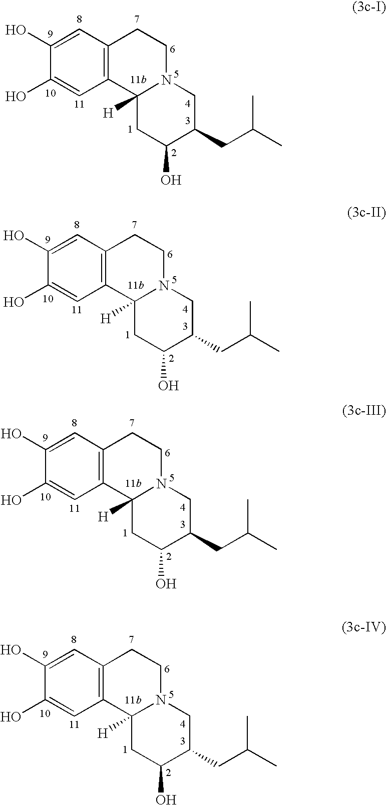

and/or pharmaceutically acceptable salts thereof.

Each of the compounds of the invention as hereinbefore defined may be provided in substantially pure form, for example at an isomeric purity of greater than 90%, typically greater than 95% and more preferably greater than 98%, or greater than 99% or greater than 99.5% or greater than 99.9%.

In the context of an individual isomer, the term “isomeric purity” refers to the amount or concentration of a particular isomer of a compound in question relative to the total amount or concentration of all isomeric forms of the compound.

In the context of a group of compounds (e.g., formulae (1a), (2b) and 3(b)), the term “isomeric purity” refers to the amount or concentration of compound falling within the said group expressed as a percentage relative to the total amount or concentration of the said compound and any isomeric forms thereof falling outside the group.

In this application, unless the context requires otherwise, a reference to a compound of the formula (1) includes not only formula (1) but also formulae (1a), (2), (2a), (2b), (2c), (3), (3a), (3b), (3c), subgroups thereof and particular compounds within the sub-groups as hereinbefore defined, as well as the compounds (2a-I), (2a-II), (2c-I) and (2c-II). The aforementioned compounds may also be referred to for convenience as the compounds of the invention.

Thus, another aspect of the invention is a composition that includes any of the compounds described herein and a pharmaceutically acceptable carrier. For example, the compositions can include any compound of formula (1):

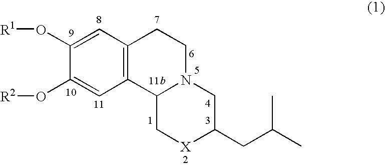

or a pharmaceutically acceptable salt or tautomer thereof;
wherein R¹ and R² are each selected from hydrogen and methyl; and
X is CHOH or C═O; and a pharmaceutically acceptable carrier.

Such compositions can include any of the compounds described herein. However, in some embodiments, certain compounds may be excluded from the compositions of the invention, for example, as described above. For example, in some embodiments, the pharmaceutical compositions described herein can include a compound of the formula (1):

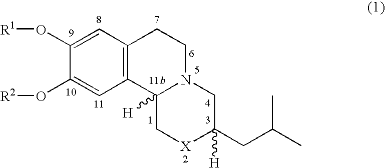

or a pharmaceutically acceptable salt or tautomer thereof;
wherein R¹ and R² are each selected from hydrogen and methyl, provided that at least one of R¹ and R² is hydrogen; and X is CHOH or C═O;
and a pharmaceutically acceptable carrier.

The compounds and compositions described herein can include pharmaceutically acceptable salts, as described in more detail below.

### Pharmaceutically Acceptable Salts

A reference to a compound of formula (1) also includes within its scope not only the free base of the compound but also its salts, and in particular acid addition salts.

Particular acids from which the acid addition salts are formed include acids having a pKa value of less than 3.5 and more usually less than 3. For example, the acid addition salts can be formed from an acid having a pKa in the range from +3.5 to −3.5.

Preferred acid addition salts include those formed with sulphonic acids such as methanesulphonic acid, ethanesulphonic acid, benzene sulphonic acid, toluene sulphonic acid, camphor sulphonic acid and naphthalene sulphonic acid.

One particular acid from which acid addition salts may be formed is methanesulphonic acid.

Salts can be prepared by the methods described herein or conventional chemical methods such as the methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free base form of the compound with the appropriate base or acid in water, or in an organic solvent, or in a mixture of the two. In some embodiments, non-aqueous media such as ether, ethyl acetate, ethanol, isopropanol, or acetonitrile are used.

Salts may also be formed with a hydroxyl group on the benzene ring of the 1,3,4,6,7,11b-hexahydro-benzo(a)quinolizine structure. The salts can be formed by reaction of the compound with a base such as an alkali metal hydroxide (e.g. sodium hydroxide or potassium hydroxide) or alkaline earth metal hydroxide, ammonia or a primary secondary or tertiary amine.

The salts are typically pharmaceutically acceptable salts. However, salts that are not pharmaceutically acceptable may also be prepared as intermediate forms which may then be converted into pharmaceutically acceptable salts. Such non-pharmaceutically acceptable salt forms also form part of the invention.

### Methods for the Preparation of Compounds of the Formula (1)

Compounds of the formula (1) can be prepared by the methods set out below or methods analogous thereto.

Compounds of the formula (1) wherein X is C═O, R¹ is hydrogen and R² is methyl (i.e. compounds of the formula (2a)) can be prepared by the sequence of reactions set out in Scheme 1.

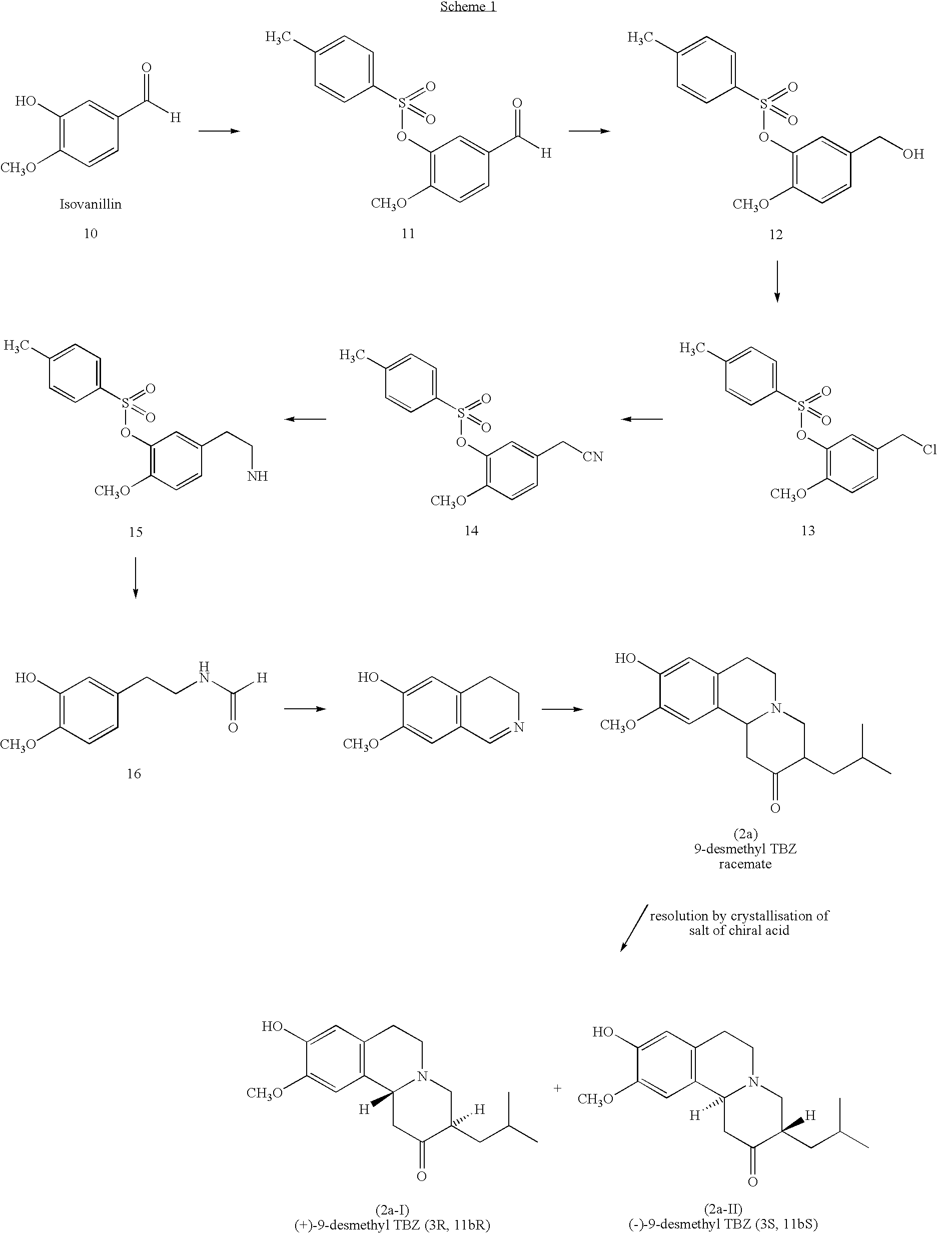

In Scheme 1, isovanillin 10 is tosylated using tosyl chloride and pyridine in dichloromethane or another chlorinated solvent to give tosyl isovanillin 11. The tosyl vanillin 11 is then reduced to the corresponding alcohol 12 using sodium borohydride in a dichloromethane/methanol (10:1) solvent. Treatment of the alcohol 12 with neat thionyl chloride followed by heating to about 60° C. gives the benzyl chloride compound 13. The benzyl chloride compound 13 is then converted to the nitrile 14 by reaction with potassium cyanide in acetonitrile in the presence of 18 crown 6 ether at reflux temperature. The nitrile is converted to the substituted phenylethylamine 15 by reduction using borane in tetrahydrofuran (THF). The tosyl protecting group on the oxygen atom attached to the meta-position on the substituted phenylethylamine 15 is removed by treatment with sodium hydroxide in ethanol/water (1:1) with heating to about 90° C. The amino group of the substituted phenylethylamine 15 is then formylated by reaction with methyl formate at room temperature to give the N-formylphenylethylamine 16. Cyclisation of the N-formylphenylethylamine 16 is carried out under Bischler-Napieralski conditions using POCl₃ in acetonitrile to give the 3,4-dihydroisoquinoline 17. The 3,4-dihydroisoquinoline 17 is then reacted with 3-(N,N-dimethylaminomethyl)-5-methyl-2-hexanone in aqueous basic conditions (˜pH 8) at room temperature to give compound (2a) as a mixture of isomers. The individual isomers can be separated by chiral chromatography or forming a chiral salt with a chiral acid such as (+) or (−) camphorsulphonic acid followed by fractional recrystallisation from a solvent such as ethanol.

Compounds of the formula (1) wherein X is C═O, R¹ is methyl and R² is hydrogen (i.e. compounds of the formula (2b)) can be prepared by the sequence of reactions set out in Scheme 2.

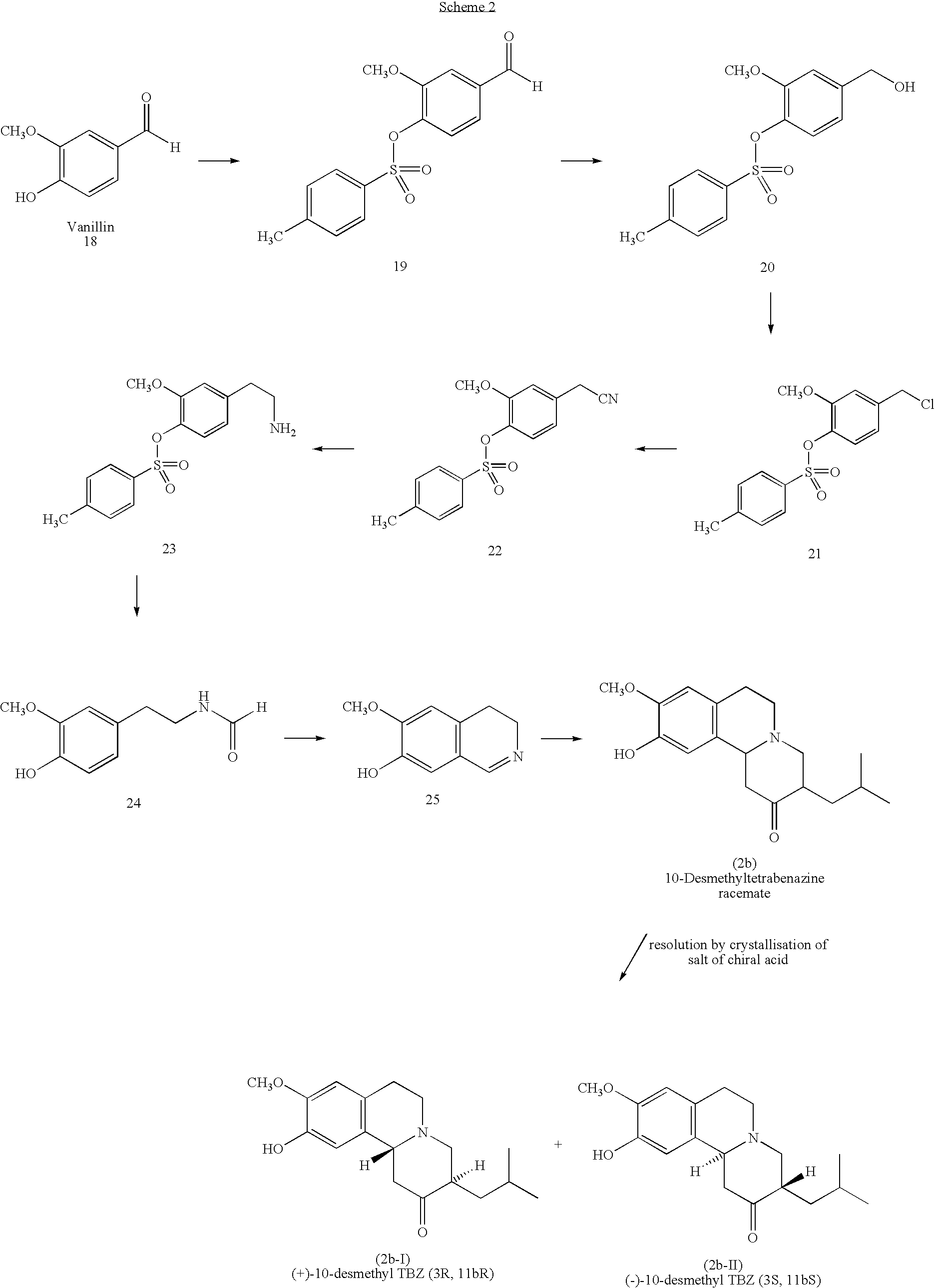

The reaction conditions used in Scheme 2 are broadly the same as the conditions used in Scheme 1 except that the starting material for the synthesis is vanillin 18 rather than isovanillin 10. Thus the vanillin 18 is tosylated using tosyl chloride and pyridine in dichloromethane to give tosyl vanillin 19. The tosyl vanillin 19 is reduced to the corresponding benzyl alcohol 20 using sodium borohydride and the resulting benzyl alcohol 20 is converted to the benzyl chloride 21 by reaction with neat thionyl chloride. The benzyl chloride compound 21 is then converted to the nitrile 22 by reaction with potassium cyanide and the nitrile 22 in turn is converted to the substituted phenylethylamine 23 by reduction using borane. Removal of the tosyl protecting group from the substituted phenylethylamine 23 followed by formylation using methyl formate gives the N-formylphenylethylamine 24 which can then be cyclised using POCl₃ in acetonitrile to give the 3,4-dihydroisoquinoline 25. The 3,4-dihydroisoquinoline 25 is then reacted with 3-(N,N-dimethylaminomethyl)-5-methyl-2-hexanone to give compound (2b) as a mixture of isomers which can be separated into the individual isomers (2b-I) and (2b-II) by chiral chromatography or chiral salt formation followed by fractional recrystallisation.

Compounds of the formula (1) wherein X is C═O, and R¹ and R² are both hydrogen (i.e. compounds of the formula (2c)) can be prepared by the sequence of reactions set out in Scheme 3. The reaction conditions used in Scheme 3 are broadly similar to those employed in Schemes 1 and 2 as described above.

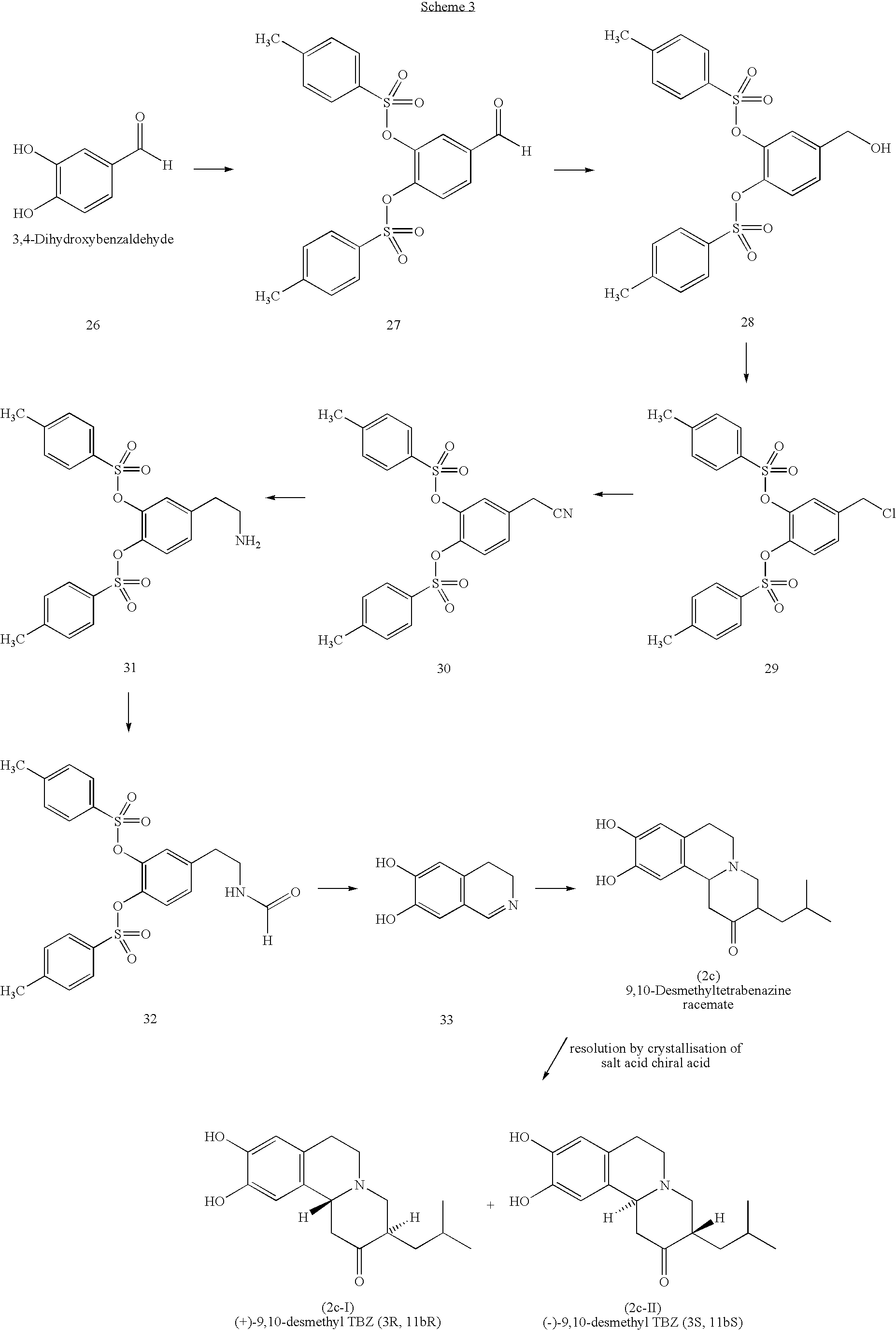

Compounds of the formulae (3A-I) and (3A-II) can be prepared by the synthetic routes shown in Scheme 4.

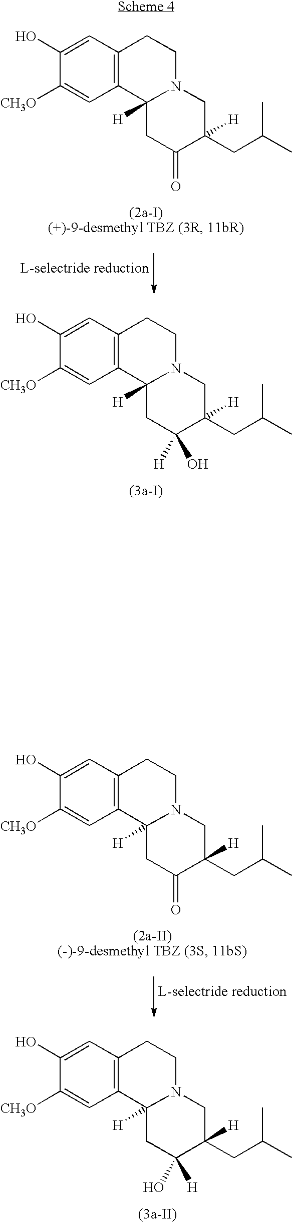

In Scheme 4, the RR and SS isomers of 9-desmethyltetrabenazine are reduced to give the (β-isomers (2,3-cis isomers) of 9-desmethyldihydroterabenazine by using the stereoselective reducing agent L-selectride.

Compounds of the formulae (3a-III) and 3a-IV) can be prepared either by the methods described in Kilbourn et al., Chirality, 9:59-62 (1997), or by the synthetic routes shown in Scheme 5.

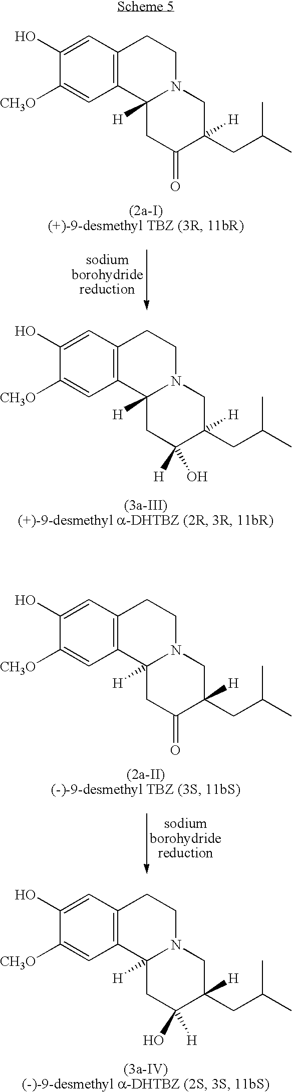

In Scheme 5, sodium borohydride is used instead of L-selectride and the 9-desmethyltetrabenazine isomers (2a-I) and (2a-II) are reduced to give the 9-desmethyldihydrotetrabenazine isomers (3a-III) and (3a-IV) respectively.

The compound of formula (3a-V) can be prepared by the route shown in Scheme 6.

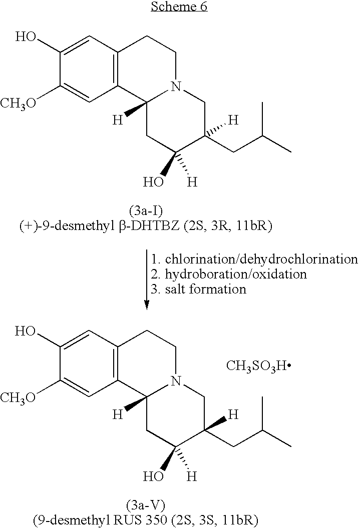

The starting material for Scheme 6 is compound (3a-I) which is prepared according the methods illustrated in Scheme 4. Compound (3a-I) is dehydrated to form an intermediate alkene (not shown) by chlorination/dehydrochlorination using phosphorus pentachloride in a chlorinated solvent such as dichloromethane followed by treatment with a base such as sodium carbonate. The intermediate alkene is then stereoselectively rehydrated by employing a hydroboration/oxidation procedure using borane-THF in tetrahydrofuran (THF) to form a borane intermediate (not shown) which is then oxidised with hydrogen peroxide in the presence of a base such as sodium hydroxide. The chlorination/dehydrochlorination and hydroboration/oxidation reactions may be carried out using conditions analogous to those described in the examples of our earlier application WO2005/077946, the contents of which are incorporated herein by reference.

The compound of formula (3a-VI) can be prepared by the route shown in Scheme 7 wherein the chlorination/dehydrochlorination, and hydroboration/oxidation steps are carried out under the same general conditions as described in Scheme 6.

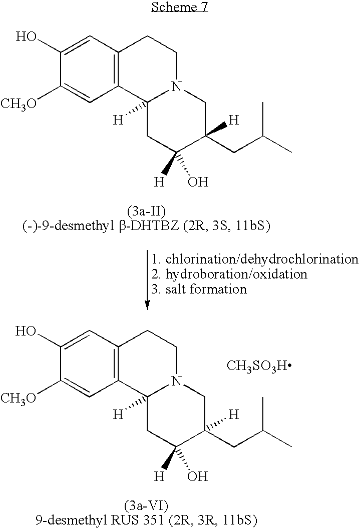

In a variation of the reaction sequence shown in Schemes 6 and 7, the intermediate alkene formed by dehydration of the compound of formulae (3a-I) and (3a-II) can be converted to an epoxide. The epoxidation reaction can be carried out using conditions and reagents well known to the skilled chemist, see for example J. March, Advanced Organic Chemistry, 4^{th} Edition, John Wiley & Sons, New York, 1992, pages 826-829 and references therein. Typically, a per-acid such as meta-chloroperbenzoic acid (MCPBA), or a mixture of a per-acid and a further oxidising agent such as perchloric acid, may be used to bring about epoxidation.

The epoxide can subsequently be subjected to ring opening by reaction with borane-THF in a polar non-protic solvent such as ether (e.g. tetrahydrofuran) at ambient temperature, followed by heating in the presence of water, sodium hydroxide and hydrogen peroxide at reflux temperature, to give the compounds (3a-VII) and (3a-VIII) wherein the 2- and 3-substituents have a cis relative configuration.

The compounds (3b-I) and (3b-II) can be prepared can be prepared by the L-selectride reduction of compounds (2b-I) and (2b-II) respectively shown in Scheme 8.

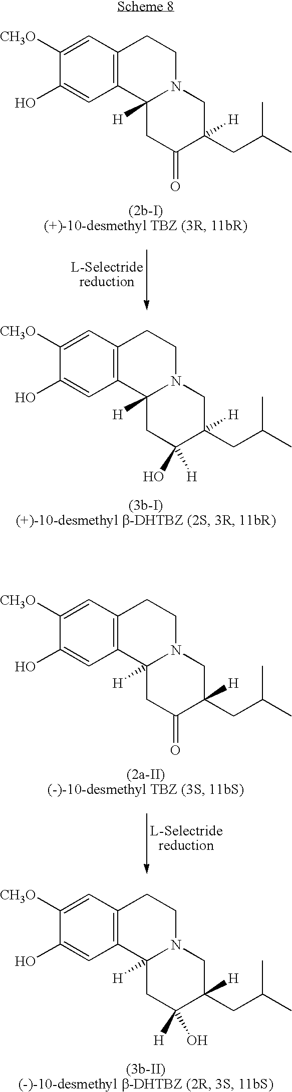

The compounds (3b-III) and (3b-IV) can be prepared by the sodium borohydride reduction of compounds (2b-I) and (2b-II) respectively as shown in Scheme 9.

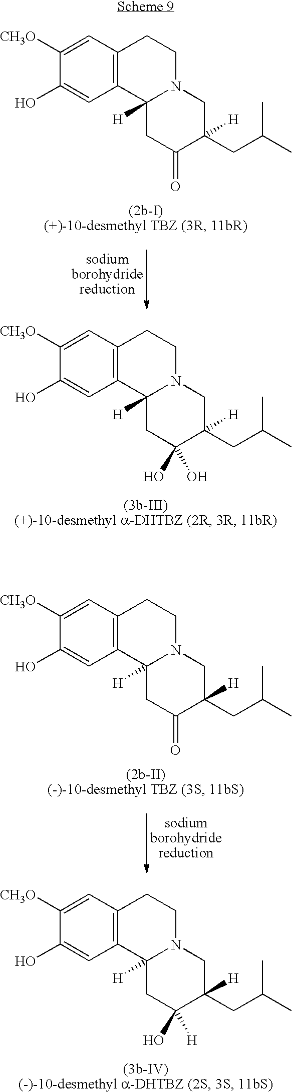

The compound (3b-VII) can be prepared from compound (3b-I) by a chlorination-dehydrochlorination-hydroboration-oxidation reaction sequence as shown in Scheme 10 using conditions analogous to those described in Scheme 6.

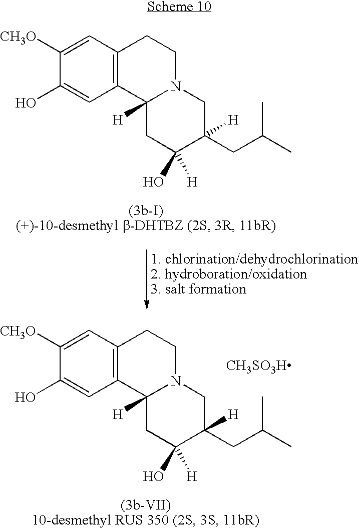

The compound (3b-VIII) can be prepared from compound (3b-II) by the chlorination-dehydrochlorination-hydroboration-oxidation reaction sequence shown in Scheme 11 using the conditions described in Scheme 6.

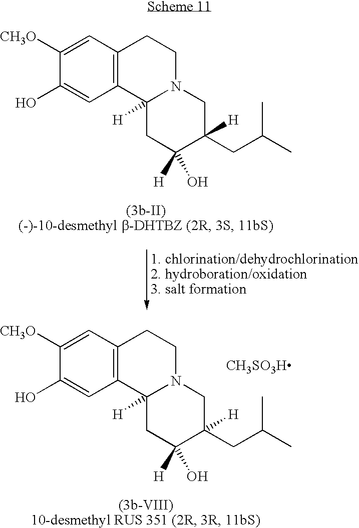

The compounds (3b-V) and (3b-VI), in which the 2- and 3-substituents are in a cis relative configuration, can be prepared by subjecting the compounds (3b-I) and (3b-II) to chlorination/dehydrochlorination to give an intermediate alkene which is then oxidised to the epoxide and ring opened by treatment with borane, under the conditions described above for the preparation of (3a-VII) and (3a-VIII).

The compounds (3c-I) and (3c-II) can be prepared from compounds (2c-I) and (2c-II) respectively by reduction with L-selectride as shown in Scheme 12.

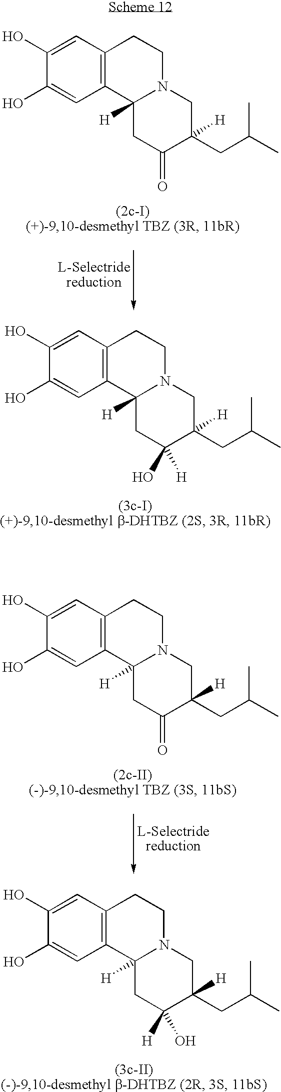

Compounds (3c-III) and (3c-IV) can be prepared from compounds (2c-I) and (2c-II) respectively by sodium borohydride reduction as shown in Scheme 13.

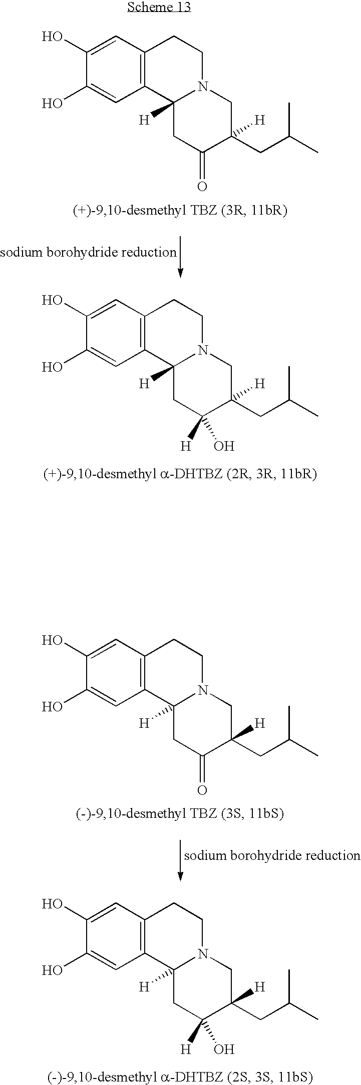

The compounds (3c-V), (3c-VI), (3c-VII) and (3c-VIII), in which the hydrogen atoms at the 3 and 11b positions are in the cis relative orientation, can be prepared by the chlorination/dehydrochlorination procedures described above to give an intermediate alkene followed by either hydroboration/oxidation to give compounds (3c-VII) and (3c-VIII) or epoxidation/diborane treatment to give the compounds of formulae (3c-V) and (3c-VI).

In a variation on the synthetic route shown in Scheme 11, the compound (3b-VIII) in the form of its mesylate salt can be prepared from the (−) isomer of 10-desmethyltetrabenazine as shown in Scheme 14. The (−) isomer can be prepared by the resolution of racemic 10-desmethyltetrabenazine as described in Example 7 below.

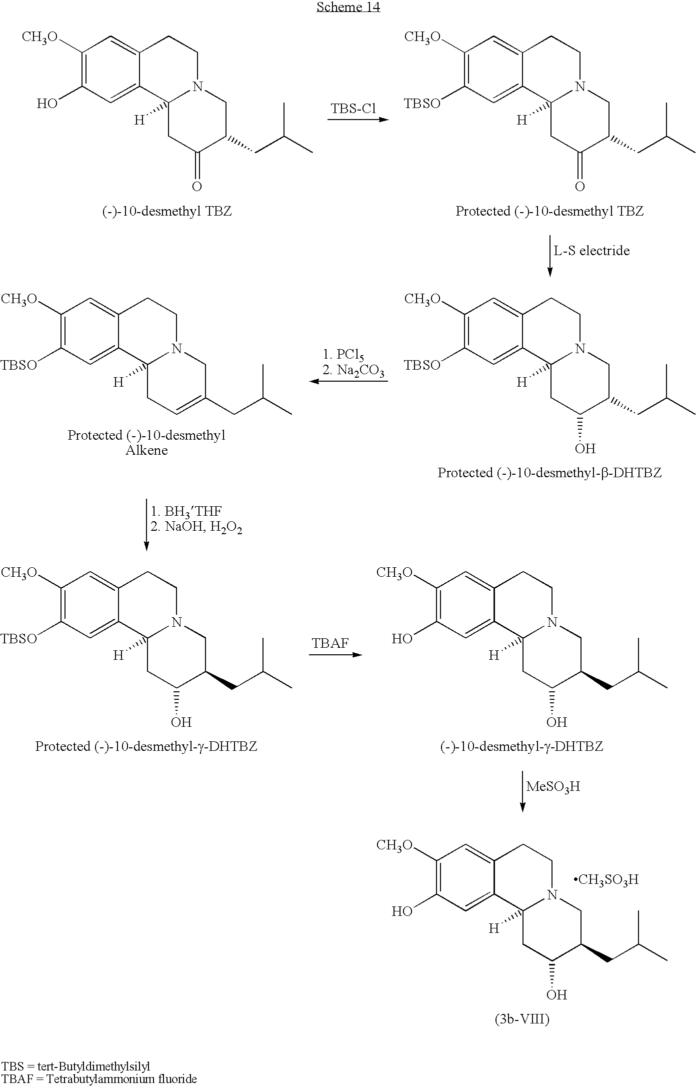

In Scheme 14, the 10-hydroxyl group of (−)-10-desmethyltetrabenazine is protected by reaction with tert-butyldimethylsilyl chloride in the presence of a base such as imidazole. The reaction is typically carried out in a polar non-protic solvent such as dimethylformamide. The resulting protected (−)-10-desmethyltetrabenazine is then reduced with L-selectride to give the (−)-10-desmethyldihydrotetrabenazine which is then chlorinated and dehydrochlorinated (PCl₅ followed by Na₂CO₃) as described above to give a protected (−)-10-desmethyltetrabenazine alkene intermediate. Treatment of the alkene intermediate with borane in THF followed by alkaline hydrogen peroxide gives the protected (−)-10-desmethyldihydrotetrabenazine in which the hydrogen atoms in the 3 and 11b positions are in a cis relative configuration. Removal of the tert-butyldimethylsilyl by reaction with tetrabutylammonium fluoride gives the compound of formula (3b-VIII) which can be converted to the mesylate salt by treatment with methanesulphonic acid.

The preparation of compounds (3b-VII) and (3b-VIII) from (±)-10-desmethyltetrabenazine can be carried out using the sequence of reactions shown in Scheme 15.

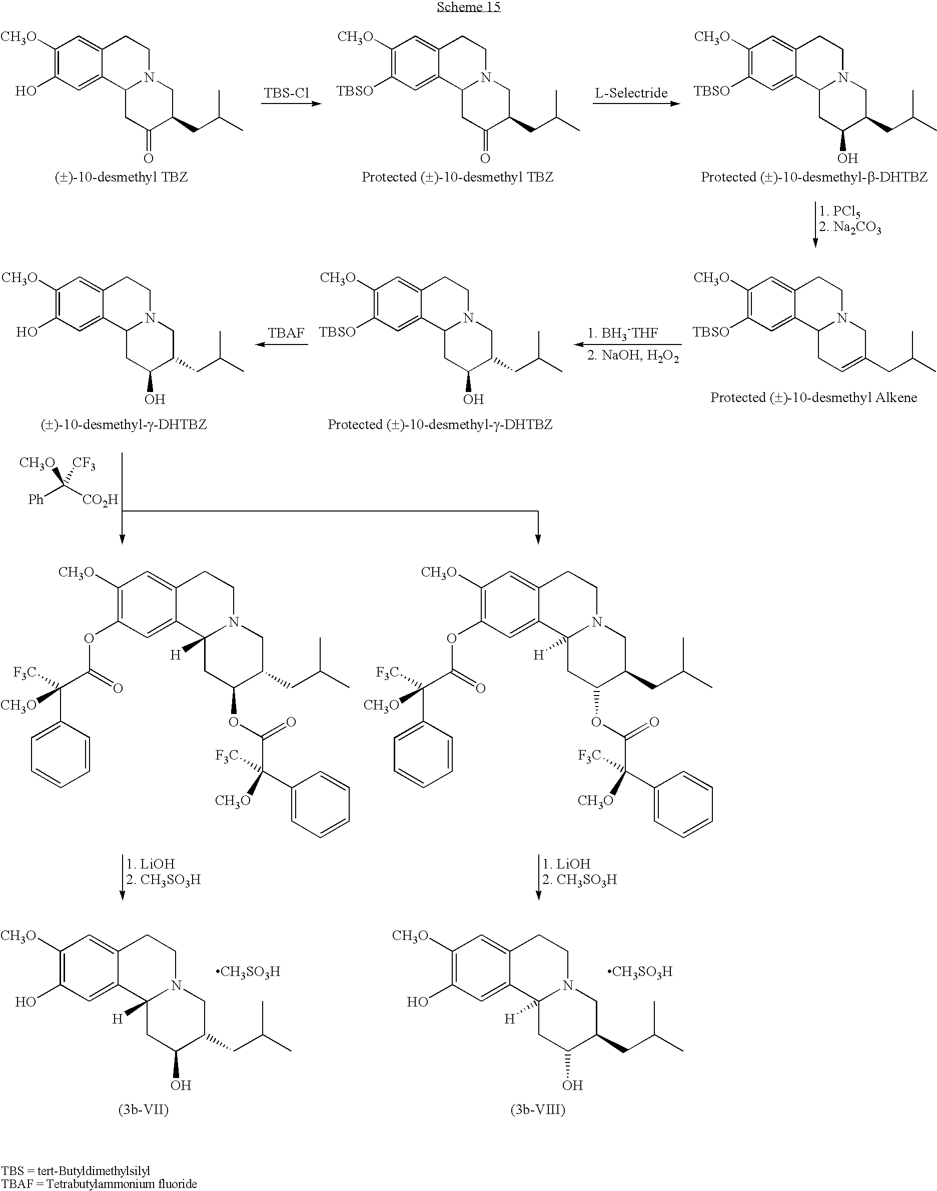

In Scheme 15, (±)-10-desmethyltetrabenazine is treated with tert-butyldimethylsilyl chloride and imidazole in dimethylformamide to convert the 10-hydroxyl group to a protected form (tert-butyldimethylsilyloxy group). Conversion to an alkene intermediate using the chlorination/dehydrochlorination procedure described above followed by treatment with borane and alkaline hydrogen peroxide and then deprotection using tetrabutylammonium fluoride gives a mixture of the enantiomers (3b-VII) and (3b-VIII).

Separation of the two enantiomers can be carried out by esterifying the 3-hydroxyl group of the 10-desmethyldihydrotetrabenazine with an optically active form of Mosher's acid, such as the R (+) isomer shown below, or an active form thereof:

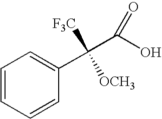

The resulting esters of the two enantiomers of the 10-desmethyldihydro-tetrabenazinee can then be separated by chromatography (e.g. HPLC) and the separated esters hydrolysed to give the individual isomers (3b-VII) and (3b-VIII) using a base such as an alkali metal hydroxide (e.g. LiOH) in a polar solvent such as methanol.

The acid addition salts of each enantiomer can then be prepared where desired by treatment with an acid such as methanesulphonic acid.

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999). A hydroxy group may be protected, for example, as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc). In particular, it may be necessary or desirable to protect one or more of the hydroxyl groups present to prevent unwanted side reactions from taking place. For example, in the case of reactions involving compounds having hydroxyl groups at the 9- and 10-positions, particularly where the reactions involve oxidizing conditions, it may be desirable to protect the hydroxyl groups, e.g. example by forming an acetonide derivative, or a dibenzyl derivative or a mono- or di-tosyl derivative.

Once formed, the compounds of the invention can be purified by standard methods such as recrystallisation and chromatography.

### Biological Properties and Therapeutic Uses

It is envisaged that the compounds of the invention will be useful in the prophylaxis or treatment of a variety of different disease states and conditions.

Accordingly, in another aspect, the invention provides a compound of the formula (1) as defined herein, or a pharmaceutically acceptable salt thereof, for use in a pharmaceutical composition or medicine.

In another aspect, the invention provides a compound of the formula (1) as defined herein, or a pharmaceutically acceptable salt thereof, for use in the treatment of movement disorders.

The movement disorders can be, for example, hyperkinetic movement disorders such as Huntington's disease, hemiballismus, senile chorea, tic disorders, tardive dyskinesia, dystonia, myoclonus and Tourette's syndrome.

Of particular interest in the treatment of hyperkinetic movement disorders are the compounds 2a-I, 2b-I, 2c-I, 3a-I, 3b-I, 3c-I, 3a-V, 3b-VII and 3c-VII, or pharmaceutically acceptable salts thereof.

The compounds of the invention may be used to slow down or halt the progression of Huntington's disease, or to slow down or halt the development of the symptoms of Huntington's disease.

Of particular interest for use in slowing down or halting the progression of Huntington's disease, or slowing down or halting the development of the symptoms of Huntington's disease are the compounds 3a-V, 3b-VII and 3c-VII, or pharmaceutically acceptable salts thereof.

It is also envisaged that the compounds of the invention may be useful in the treatment of depression.

In a further aspect, the invention provides a compound of the formula (1) as defined herein, or a pharmaceutically acceptable salt thereof, for use in the treatment of inflammatory diseases.

Examples of inflammatory diseases include, but are not limited to, rheumatoid arthritis, osteoarthritis, traumatic arthritis, gouty arthritis, rubella arthritis, psoriatic arthritis, and other arthritic conditions; acute or chronic inflammatory disease states such as the inflammatory reaction induced by endotoxin or inflammatory bowel disease; Reiter's syndrome, gout, rheumatoid spondylitis, chronic pulmonary inflammatory disease (e.g. chronic obstructive pulmonary disease (COPD)), Crohn's disease and ulcerative colitis.

Particular inflammatory diseases and conditions are those that are sensitive to sigma receptor ligands, for example, sigma receptor antagonists.

One particular inflammatory disease is rheumatoid arthritis.

In another aspect, the invention provides a compound of the formula (1), or a pharmaceutically acceptable salt thereof, for treating, or a pharmaceutically acceptable salt thereof, for use in treating asthma.

In another aspect, the invention provides a compound of the formula (1), or a pharmaceutically acceptable salt thereof, for use in treating multiple sclerosis or an autoimmune myelitis.

The treatment of multiple sclerosis may consist of or comprise any one or more of:
halting the progression of the disease;
slowing the progression of the disease;
modifying the progression of the disease;
providing symptomatic relief, e.g. by eliminating or reducing the severity of one or more symptoms;
extending periods of remission;
preventing relapses;
reducing the severity of relapses; and
preventing or slowing the progression from an initial period of relapsing-remitting MS to secondary progressive MS.

The treatment may consist of or comprise the elimination, amelioration or reduction in severity of any one or more symptoms of multiple sclerosis, in any combination, selected from:
weakness and/or numbness in one or more extremities;
tingling of the extremities;
tight band-like sensations around the trunk or limbs;
tremor of one or more extremities;
dragging or poor control of one or both legs;
spastic or ataxic paraparesis;
paralysis of one or more extremities;
hyperactive tendon reflexes;
disappearance of abdominal reflexes;
Lhermitte's sign;
retrobulbar or optic neuritis;
unsteadiness in walking;
problems with balance,
increased muscle fatigue;
brain stem symptoms (diplopia, vertigo, vomiting);
disorders of micturition;
hemiplegia;
trigeminal neuralgia;
other pain syndromes;
nystagmus and ataxia;
cerebellar-type ataxia;
Charcot's triad; diplopia;
bilateral internuclear opthalmoplegia;
myokymia or paralysis of facial muscles;
deafness;
tinnitus;
unformed auditory hallucinations (because of involvement of cochlear connections);
transient facial anesthesia or of trigeminal neuralgia;
urinary and/or faecal incontinence
bladder dysfunction euphoria;
depression;
fatigue;
dementia;
dull, aching pain in the low back;
sharp, burning, poorly localized pains in a limb;
abrupt attacks of neurologic deficit;
dysarthria and ataxia;
paroxysmal pain and dysesthesia in a limb;
flashing lights;
paroxysmal itching;
tonic seizures;
changes in sensation;
visual problems;
muscle weakness;
difficulties with coordination and speech;
cognitive impairment;
overheating; and
impaired mobility and disability.

Compounds of particular interest in the treatment of inflammatory diseases, multiple sclerosis and asthma are compounds 3a-V, 3b-VII and 3c-VII, or pharmaceutically acceptable salts thereof.

The invention also provides a compound of the formula (1) as defined herein, or a pharmaceutically acceptable salt thereof, for use in the treatment of psychoses.

Thus, the compounds of the invention may be used to prevent, alleviate or reduce any one or more psychotic episodes, psychoses or symptoms selected from:
delusions;
hallucinations;
visual hallucinations;
auditory hallucinations;
hallucinations involving tactile sensations, tastes or smells;
confusion;
emotional, behavioral, or intellectual disturbances;
withdrawal from reality;
illogical and/or disorganized patterns of thinking;
paranoid or delusional beliefs;
paranoia
grandiose delusions;
persecutory or self-blaming delusions; and
personality changes.

The psychotic episodes, psychoses or symptoms prevented, alleviated or reduced in accordance with the invention may be any one or more selected from those arising from or associated with:
psychosis caused by or associated with schizophrenia;
psychosis caused by or associated with bipolar disorder (manic depression);
psychosis caused by or associated with severe clinical depression;
psychosis induced by disorders and conditions such as:
   electrolyte disorder;
   urinary tract infections in the elderly;
   pain syndromes;
   drug toxicity;
   drug withdrawal; and
   infections of or injuries to the brain;
psychosis caused by chronic psychological stress (brief reactive psychosis);
psychosis triggered or exacerbated by severe mental stress; and
psychosis triggered by or arising from or following illnesses and conditions such as AIDS, leprosy, malaria and mumps.

In one embodiment, the symptoms or psychoses arise from or are associated with schizophrenia and may be any one or more symptoms selected from:
delusions;
hallucinations;
confusion;
emotional, behavioral, or intellectual disturbances;
withdrawal from reality; and
illogical patterns of thinking.

In a further aspect, the invention provides a compound of the formula (1) as hereinbefore defined, or a pharmaceutically acceptable salt thereof, for use in treating a cognitive deficit associated with schizophrenia.

In another aspect, the invention provides a compound of the formula (1) as defined herein, or a pharmaceutically acceptable salt thereof, for use in treating anxiety.

In a further aspect, the invention provides a compound of the formula (1), or a pharmaceutically acceptable salt thereof, for treating a cognitive deficit in a patient.

In another aspect, the invention provides a compound of the formula (1), or a pharmaceutically acceptable salt thereof, for use in treating dementia.

The dementia may be, for example, dementia associated with Alzheimer's disease, or Lewy body dementia, or dementia arising from injury to the cerebrovascular system (e.g. stroke).

The compounds of the invention may be used to treat cognitive deficits in patients suffering from dementia.

Compounds of particular interest for the treatment of psychoses, dementia and cognitive deficits are the compounds 3a-VI, 3b-VIII and 3c-VIII, or pharmaceutically acceptable salts thereof.

In a further aspect, the invention provides a compound of the formula (1), or a pharmaceutically acceptable salt thereof, for use in the treatment of diabetes mellitus.

The invention also provides a compound of the formula (1), or a pharmaceutically acceptable salt thereof, for use in (i) enhancing glucose dependent insulin secretion in a patient; and/or (ii) enhancing glucose tolerance in a patient.

The invention further provides the use of a compound of the formula (1), or a pharmaceutically acceptable salt thereof, as defined herein for the manufacture of a medicament for the prophylaxis or treatment of any one or more of the diseases or conditions listed above.

The invention also provides a method for the treatment of any one or more of the diseases or conditions listed above, which method comprises administering to a patient (e.g. a mammalian patient such as a human) a therapeutically effective (preferably non-toxic) amount of a compound of the formula (1) or a pharmaceutically acceptable salt thereof.

In treating each of the conditions listed above, the compounds of the invention will generally be administered to a subject in need of such administration, for example a human or animal patient, preferably a human.

The compounds will typically be administered in amounts that are therapeutically or prophylactically useful and which generally are non-toxic. However, in certain situations, the benefits of administering a compound of the invention may outweigh the disadvantages of any toxic effects or side effects, in which case it may be considered desirable to administer compounds in amounts that are associated with a degree of toxicity.

A typical daily dose of the compound can be in the range from 0.025 milligrams to 5 milligrams per kilogram of body weight, for example up to 3 milligrams per kilogram of bodyweight, and more typically 0.15 milligrams to 5 milligrams per kilogram of bodyweight although higher or lower doses may be administered where required.

By way of example, an initial starting dose of 12.5 mg may be administered 2 to 3 times a day. The dosage can be increased by 12.5 mg a day every 3 to 5 days until the maximal tolerated and effective dose is reached for the individual as determined by the physician. Ultimately, the quantity of compound administered will be commensurate with the nature of the disease or physiological condition being treated and the therapeutic benefits and the presence or absence of side effects produced by a given dosage regimen, and will be at the discretion of the physician.

### Pharmaceutical Formulations

The invention also provides compounds as hereinbefore defined in the form of pharmaceutical compositions.

The pharmaceutical compositions can be in any form suitable for oral, parenteral, topical, intranasal, intrabronchial, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery.

Pharmaceutical dosage forms suitable for oral administration include tablets, capsules, caplets, pills, lozenges, syrups, solutions, sprays, powders, granules, elixirs and suspensions, sublingual tablets, sprays, wafers or patches and buccal patches.

Pharmaceutical compositions containing the dihydrotetrabenazine compounds of the invention can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, e.g.; lactose, sucrose, sorbitol or mannitol; and/or a non-sugar derived diluent such as sodium carbonate, calcium phosphate, talc, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g.; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example, a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract.

Compositions for topical use include ointments, creams, sprays, patches, gels, liquid drops and inserts (for example, intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped mouldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the inventions will generally be presented in unit dosage form and, as such, will typically contain sufficient compound to provide a desired level of biological activity. For example, a formulation intended for oral administration may contain from 2 milligrams to 200 milligrams of active ingredient, more usually from 10 milligrams to 100 milligrams, for example, 12.5 milligrams, 25 milligrams and 50 milligrams.

The active compound will be administered to a patient in need thereof (for example a human or animal patient) in an amount sufficient to achieve the desired therapeutic effect.

### EXAMPLES

The following non-limiting examples illustrate the synthesis and properties of the dihydrotetrabenazine compounds of the invention.

In the examples, the compounds prepared were characterised by NMR, analytical HPLC and mass spectroscopy (MS, ES⁺).

Mass spectra were obtained using a Platform II instrument

NMR spectra were obtained using a Varian Mercury 300 MHz instrument using CD₃OD as solvent.

HPLC analysis was carried out using the following conditions:

| Column: | Prodigy ODS(3) 5 μm 100 Å 250 × 4.6 mm | | |
|---|---|---|---|
| Mobile phase A: | 10 mM ammonium acetate solution (pH 8.0) | | |
| Mobile phase B: | acetonitrile:10 mM ammonium acetate solution | | |
| | (pH 8.0) (9:1 v/v) | | |
| | time (min) | A (%) | B (%) |
| Gradient | 0.0 | 90 | 10 |
| | 30.0 | 30 | 70 |
| | 35.0 | 10 | 90 |
| | 40.0 | 10 | 90 |
| | 40.1 | 90 | 10 |
| | 45.0 | 90 | 10 |

| | |
|---|---|
| Flow: | 1.0 ml min⁻¹ |
| Temperature: | 25° C. |
| UV: | 235 nm |
| Inj.: | 10 μl, 1 mg ml⁻¹ in water:acetonitrile (1:1 v/v) |

### Example 1

### 9-Desmethyltetrabenazine

### 1A. Preparation of 4-Methoxy-3-tosyloxybenzaldehyde

Isovanillin (20.17 g, 0.13 mol) (Aldrich, 07814AC), tosyl chloride (26.24 g, 0.14 mol), and tetrabenzylammonium chloride (80 mg, 0.35 mmol) were dissolved in dichloromethane (200 ml) with stirring at room temperature. A solution of sodium hydroxide (5.62 g, 0.14 mol) in water (50 ml) was added to the stirred reaction and the organic phase which separated was observed to turn yellow. The reaction mixture was stirred for three days at room temperature. The reaction aqueous layer was separated and the organic layer was washed with 2M aqueous hydrochloric acid (50 ml), dried over anhydrous magnesium sulphate, filtered and concentrated at reduced pressure to give a pale brown solid (40.2 g). This was identified as 4-methoxy-3-tosyloxybenzaldehyde from its ¹H-NMR spectrum.

### 1B. 4-Methoxy-3-tosyloxybenzyl alcohol

4-Methoxy-3-tosyloxybenzaldehyde (10.0 g, 33 mmol) and sodium borohydride (1.37 g, 36 mmol) were stirred together in a mixture of dichloromethane:methanol (10:1) (50 ml) at room temperature. Care was taken to ensure that the ratio of dichloromethane to methanol in the solvent mixture did not exceed 10 to 1, because unwanted side products are formed at higher ratios. The reaction mixture was stirred at room temperature and TLC analysis [silica, eluting with ethyl acetate:hexane (1:1)] of an aliquot of the reaction mixture after two hours showed no starting material remained. The reaction mixture was concentrated to dryness at reduced pressure and the residue dissolved in dichloromethane (100 ml). The organic solution was washed with water (100 ml), dried over anhydrous potassium carbonate, filtered and concentrated at reduced pressure to give a solid, (10.0 g). This was identified as 4-Methoxy-3-tosyloxybenzyl alcohol from its ¹H-NMR spectrum

### 1C. 4-Methoxy-3-tosyloxybenzyl chloride

In a flask pre-cooled to 0° C., 4-methoxy-3-tosyloxybenzyl alcohol (7.8 g, 25.32 mmol) was treated with thionyl chloride (2.2 ml, 30.39 mmol), which was added dropwise with stirring to form a solution. The reaction mixture was stirred and heated at 60° C. and TLC analysis [silica, eluting with ethyl acetate:hexane (1:1)] of an aliquot of the reaction mixture after two hours showed no starting material remained. The reaction mixture was concentrated at reduced pressure and the residue taken up in dichloromethane (100 ml). The organic solution was washed with water (2×50 ml), dried over anhydrous potassium carbonate, filtered and concentrated once more at reduced pressure to a solid residue (7.69 g). This was identified as 4-Methoxy-3-tosyloxybenzyl chloride from its ¹H-NMR spectrum.

### 1D. 4-Methoxy-3-tosyloxyphenylacetonitrile

4-Methoxy-3-tosyloxybenzyl chloride (7.70 g, 23.6 mmol) was dissolved with stirring in dry acetonitrile (250 ml) and 18-crown-6 (7.48 g, 28.3 mmol) (Alfa Aesar, 10127224) and potassium cyanide (1.85 g, 28.3) were added. The reaction mixture was stirred at reflux under an argon atmosphere for twenty four hours and appeared complete following TLC analysis [silica, eluting with ethyl acetate:hexane (1:3)] of an aliquot drawn from the reaction. The reaction mixture was allowed to cool and was then concentrated at reduced pressure. The residue was dissolved in dichloromethane (100 ml) and the solution washed with water (100 ml). The organic layer was separated, dried over potassium carbonate, filtered and concentrated at reduced pressure to give an oil which solidified on standing. This was re-crystallised from methanol to give the product (5.8 g). This was identified as 4-methoxy-3-tosyloxyphenylacetonitrile from its ¹H-NMR spectrum.

### 1E. 4-Methoxy-3-tosyloxyphenylethylamine

4-Methoxy-3-tosyloxyphenylacetonitrile (31 g, 98 mmol) was dissolved with stirring in anhydrous tetrahydrofuran (250 ml) under an argon atmosphere. A solution of borane in THF (800 ml, 1M) was added dropwise at room temperature to the stirring reaction under argon pressure via a cannula. The reaction mixture was then stirred at room temperature overnight before quenching by dropwise addition of methanol (250 ml) to the stirring mixture. The reaction mixture was then concentrated at reduced pressure and the residue taken up in a mixture of methanol (150 ml) and hydrochloric acid (375 ml, approx. 5M). The reaction mixture was allowed to stir for one hour before sodium hydroxide solution (30%) was added until the pH was >11. The resulting solution was extracted with dichloromethane (2×200 ml, 2×100 ml) and the combined organic extracts were dried over anhydrous potassium carbonate, filtered and concentrated at reduced pressure. The residue was taken up in hydrochloric acid (200 ml, 1 M) and washed with diethyl ether (3×100 ml). The aqueous layer was basified as above with sodium hydroxide solution (30%) until the pH >11 and was extracted with dichloromethane (2×200 ml, 2×100 ml). The combined organic extracts were dried over potassium carbonate, filtered and concentrated at reduced pressure to give a solid residue (28.2 g). This was identified as 4-methoxy-3-tosyloxyphenylethylamine from its ¹H-NMR spectrum and ¹³C-NMR spectrum.

### 1F. N2-(4-Methoxy-3-hydroxyphenyl)ethyl formamide

Potassium hydroxide (22 g, 392.1 mmol) was dissolved with stirring in ethanol (95%):water (1:1) (100 ml), 4-methoxy-3-tosyloxyphenylethylamine (15 g, 46.7 mmol) was added to the stirred solution and the reaction mixture was stirred and heated at 90° C. for one hour. The reaction mixture was then allowed to cool to room temperature and concentrated at reduced pressure to leave a solid residue. The residual solid was then dissolved in methyl formate (70 ml) and the solution stirred at room temperature for two hours during which time a white precipitate formed. TLC analysis [silica, eluting with methanol:dichloromethane (20:80)] of an aliquot drawn from the reaction showed no remaining starting material. The reaction mixture was then concentrated at reduced pressure to leave a solid residue which was partitioned between dichloromethane (100 ml) and water (100 ml). The organic phase was separated and washed with water (2×100 ml), dried over anhydrous potassium carbonate, filtered and concentrated once more at reduced pressure to give an oil (6.9 g) which was crystallised from dichloromethane to give the product as a solid (3.5 g). This was identified as N2-(4-methoxy-3-hydroxyphenyl)ethyl formamide from its ¹H-NMR spectrum and ¹³C-NMR spectrum.

### 1G. 7-Methoxy-3,4-dihydro-isoquinolin-6-ol

A mixture of N2-(4-methoxy-3-hydroxyphenyl)ethyl formamide, (6.05 g, 31.0 mmol) and phosphorus oxychloride (3.47 ml, 37.2 mmol) was stirred at reflux in anhydrous acetonitrile (120 ml) for three hours under an argon atmosphere. The reaction was then allowed to cool and was concentrated at reduced pressure to leave an orange oil residue which was suspended in water (20 ml). Ammonium hydroxide solution (30%, 20 ml) was added slowly to the stirred aqueous suspension which dissolved to form a solution. Silica gel was added to the solution and the mixture was then was concentrated at reduced pressure to until the silica was free-flowing. The product was purified by column chromatography [silica, eluting with methanol:dichloromethane (10:90)] and the fractions of interest (identified by TLC analysis) were combined and concentrated at reduced pressure to give a yellow solid (3.9 g). This was identified as 7-methoxy-3,4-dihydro-isoquinolin-6-ol from its ¹H-NMR spectrum.

### 1H. 9-Desmethyltetrabenazine

7-Methoxy-3,4-dihydro-isoquinolin-6-ol, (0.46 g, 2.6 mmol) was dissolved with stirring in water (50 ml) together with 3-(N,N-dimethylaminomethyl)-5-methyl-2-hexanone (0.9 g, 2.7 mmol). Sodium hydroxide solution (30%) was added dropwise to the stirred mixture until the pH of the reaction was ca. 8. The reaction mixture was then stirred at room temperature for four days, occasionally checking and adjusting the pH ca. 8 with sodium hydroxide solution (30%) if required. The reaction mixture gradually turned a brown colour as the reaction progressed. The reaction mixture was then poured into a stirred saturated solution of sodium carbonate (50 ml) and extracted with dichloromethane (4×50 ml). The combined organic layers were dried over anhydrous potassium carbonate, filtered and concentrated to give a yellow oil which was purified by column chromatography [silica, eluting with ethyl acetate:hexane (1:1)] and the fractions of interest (identified by TLC analysis) were combined and concentrated at reduced pressure to give a yellow solid (3.9 g) which was crystallised from ethyl acetate:hexane to give a white solid (0.146 g). This was identified as 9-desmethyltetrabenazine from its ¹H-NMR spectrum and ¹³C-NMR spectrum and the mass spectrum was consistent with the expected structure. HPLC analysis gave a purity of 95.7% (peak area %). HPLC-RT: 29.36 MS: M+1: 304.3

### Example 2

### 10-Desmethyltetrabenazine

### 2A. 3-Methoxy-4-tosyloxybenzaldehyde

A mixture of vanillin (101 g, 0.66 mol) (Aldrich, 026K3740), potassium carbonate (96 g, 0.69 mol) and p-toluenesulphonyl chloride (134 g, 0.69 mol) was dissolved with stirring in acetone (2 L) at room temperature under argon. A white suspension formed in the pink reaction solution and the reaction mixture was stirred overnight at room temperature. The reaction mixture consisted of a colourless solution and a white suspension and the acetone solvent was removed at reduced pressure to give a solid residue. The solid residue was taken up in dichloromethane (800 ml) with stirring and water (300 ml) was added to the mixture. The aqueous phase was removed and the organic solution was washed further with water (3×300 ml), dried over anhydrous magnesium sulphate, filtered and concentrated in vacuo to give a pale yellow solid (197.8 g). TLC analysis [silica, eluting with dichloromethane] showed no starting material remained and a single product component which was identified as 3-methoxy-4-tosyloxybenzaldehyde from its ¹H-NMR spectrum and ¹³C-NMR spectrum.

### 2B. 3-Methoxy-4-tosyloxybenzyl alcohol

3-Methoxy-4-tosyloxybenzaldehyde (197.6 g, 0.65 mol) was dissolved with stirring in methanol (750 ml) and dichloromethane (750 ml) at room temperature. Sodium borohydride (49.6 g, 1.31 mol) was added in portions to the stirred solution and the reaction mixture was stirred for a further 150 min. at room temperature. TLC analysis [silica, eluting with ethyl acetate:hexane (1:1)] of an aliquot from the reaction mixture (partitioned between water and ethyl acetate, organic layer analysed) and analysing the ethyl acetate layer showed no starting material remained. The reaction solvent was evaporated at reduced pressure and the residual slurry was taken up in dichloromethane (800 ml) and water (300 ml). The two-phase mixture was poured into a separating funnel and the aqueous layer was removed. The organic layer was washed further with water (2×150 ml), dried over anhydrous magnesium sulphate, filtered and concentrated at reduced pressure to give a pale brown solid (178.2 g). TLC analysis [silica, eluting with ethyl acetate:hexane (1:1)] showed no starting material and a single product component which was identified as 3-methoxy-4-tosyloxybenzyl alcohol from its ¹H-NMR spectrum.

### 2C. 3-Methoxy-4-tosyloxybenzyl chloride

3-Methoxy-4-tosyloxybenzyl alcohol (170 g, 0.55 mol) was cooled to 0° C. under argon using an ice bath and thionyl chloride (50 ml, 0.69 mol) was added slowly with stirring to form a solution. The reaction mixture was stirred and heated under argon at 50° C. for five hours to give a dark green solution. TLC analysis [silica, eluting with dichloromethane:hexane (3:1)] of an aliquot from the reaction mixture showed no starting material remained. The reaction solvent was removed at reduced pressure and the residual syrup taken up in dichloromethane (800 ml) before water (300 ml) was added. The two-phase reaction mixture was poured into a separating funnel and the aqueous layer was removed. The organic layer was washed further with water (2×150 ml), dried over anhydrous potassium carbonate, filtered and concentrated at reduced pressure to give a dark green syrup which was crystallised from hot methanol to give a pale yellow solid (129.0 g). TLC analysis [silica, eluting with dichloromethane:hexane (3:1)] showed no remaining starting material and a single product component which was identified as 3-methoxy-4-tosyloxybenzyl chloride from its ¹H-NMR spectrum.

### 2D. 3-Methoxy-4-tosyloxyphenylacetonitrile

A mixture of 3-methoxy-4-tosyloxybenzyl chloride (56 g, 0.17 mol), 18-crown-6 (55 g, 0.21 mol) and potassium cyanide (14 g, 0.21 mol) was dissolved with stirring in dry acetonitrile (500 ml) under argon to give a green solution with a white suspension. The reaction mixture was then stirred at reflux for six hours and allowed to cool before the solvent was removed at reduced pressure. The residual syrup was taken up in dichloromethane (1 L) and water (300 ml) was added before the mixture was poured into a separating funnel and the aqueous layer removed. The organic layer was washed further with water (2×250 ml), dried over anhydrous potassium carbonate, filtered and concentrated at reduced pressure to give a purple syrup that crystallised on standing at room temperature overnight.

The crude product was crystallised twice using hot methanol to give a pale pink solid (37.4 g). TLC analysis [silica, eluting with dichloromethane] showed no starting material remained and a single product component which was identified as 3-methoxy-4-tosyloxyphenylacetonitrile from its ¹H-NMR spectrum.

### 2E. N2-(3-Methoxy-4-hydroxyphenyl)ethyl formamide

3-Methoxy-4-tosyloxyphenylacetonitrile (35.3 g, 0.11 mol) was dissolved with stirring in dry tetrahydrofuran (250 ml) under argon at room temperature over twenty minutes. A solution of 1M borane in THF (800 ml, 0.80 mol) was added dropwise via cannula to the stirred reaction mixture over forty five minutes to give a clear yellow solution which was then was stirred at room temperature overnight. TLC analysis [silica, eluting with methanol:dichloromethane (2:8)] of an aliquot from the reaction mixture showed no starting material remained. The stirred reaction mixture was cooled to 0° C. and methanol (150 ml) was slowly added dropwise (caution: a large amount of hydrogen gas is evolved) and once the initial vigorous reaction subsided an additional quantity of methanol (350 ml) was added to ensure no borane remained. The solvent was removed at reduced pressure and the residual foam dissolved using 5 M hydrochloric acid (500 ml) and methanol (1 L). The resulting solution was stirred at room temperature for one hour before it was basified by addition of aqueous sodium hydroxide (ca. 2 M). A white precipitate was formed and the reaction mixture was poured into a separating funnel and extracted with dichloromethane (10×100 ml). The organic extracts were combined, dried over anhydrous potassium carbonate and concentrated at reduced pressure to give a brown viscous oil (33.4 g). The oil was dissolved in 1 M hydrochloric acid (500 ml), and the solution poured into a separating funnel and washed with diethyl ether (2×200 ml). The acidic aqueous layer was made basic by the addition of 1M sodium hydroxide and then extracted with dichloromethane (10×200 ml). The organic extracts were combined, dried over anhydrous potassium carbonate, filtered and concentrated at reduced pressure to give the amine intermediate as an orange oil which was used without further purification. The orange oil (23.4 g) was dissolved with stirring in ethanol (300 ml) and potassium hydroxide (22.0 g, 352.9 mmol) in water (300 ml) was added to the reaction mixture. A cloudy solution was formed which was stirred and heated to 100° C. for one hour during which time the reaction solution became clear. TLC analysis [silica, eluting with methanol:dichloromethane (2:8)] of an aliquot from the reaction mixture showed no starting material remained and the solvents were therefore removed at reduced pressure to give a yellow solid residue. The yellow solid was dissolved with stirring in methyl formate (500 ml) and the solution was stirred at room temperature for two days until TLC analysis [silica, eluting with methanol:dichloromethane (10:90)] of an aliquot from the reaction mixture showed no starting material remained. The reaction mixture was filtered and the filtrate concentrated at reduced pressure leaving a residual syrup which was dissolved in dichloromethane (500 ml) and water (200 ml). The mixture was transferred to a separating funnel and the aqueous layer was removed. The organic layer was washed further with water (2×200 ml), dried over anhydrous potassium carbonate, filtered and concentrated at reduced pressure to give crude an orange syrup (12.0 g).

The orange syrup was purified by column chromatography [silica, eluting with methanol:dichloromethane (4:96)] to give a honey coloured syrup (7.4 g). TLC analysis [silica, eluting with methanol:dichloromethane (10:90)] showed no remaining starting material and a single product component which was identified as N2-(3-methoxy-4-hydroxyphenyl)ethyl formamide from its ¹H-NMR spectrum.

### 2F. 6-methoxy-3,4-dihydro-isoquinolin-7-ol

N2-(3-Methoxy-4-hydroxyphenyl)ethyl formamide (7.4 g, 38.0 mmol) was dissolved with stirring in acetonitrile (150 ml) with gentle heating under an argon atmosphere. Phosphorous oxychloride (4.3 ml, 45.6 mmol) was added to the solution and the honey coloured reaction mixture was stirred at reflux for four hours during which time a clear dark red solution formed. TLC analysis of an aliquot from the reaction [silica, eluting with methanol:dichloromethane (10:90)] showed no starting material remained. The solvent was removed at reduced pressure and the residue was taken up in 1M hydrochloric acid (500 ml)—sonication for 10 minutes gave an orange solution and a brown precipitate. The acidic mixture was basified by addition of ammonium hydroxide (30%) and then extracted with dichloromethane (10×200 ml). The organic extracts were combined, dried over anhydrous potassium carbonate, filtered and concentrated at reduced pressure to give a yellow solid (4.3 g) which was purified by crystallisation in hot methanol to give a yellow crystalline solid (3.2 g). TLC analysis [silica, eluting with methanol:dichloromethane (10:90)] showed a single product component which was identified as 6-methoxy-3,4-dihydro-isoquinolin-7-ol from its ¹H-NMR spectrum.

### 2G. 10-Desmethyltetrabenazine

6-Methoxy-3,4-dihydro-isoquinolin-7-ol, (2.00 g, 9.95 mmol) was dissolved with stirring in water (200 ml) together with 3-(N,N-dimethylaminomethyl)-5-methyl-2-hexanone (1.87 g, 10.95 mmol). Aqueous sodium hydroxide (30%) was added dropwise to adjust the reaction to pH 8 and the reaction was then stirred at room temperature for four days. During this time the reaction pH was monitored and adjusted to pH 8 by the dropwise addition of aqueous sodium hydroxide (30%) solution if required. The reaction mixture gradually turned brown in colour. The reaction mixture was then poured into a saturated aqueous solution of sodium carbonate (200 ml) and extracted with dichloromethane (3×100 ml). The combined organic layers were dried over anhydrous potassium carbonate, filtered and concentrated to give a yellow oil which was purified using column chromatography [silica, eluting with ethyl acetate:hexane (1:1)]. The fractions of interest were identified by TLC analysis and combined and concentrated at reduced pressure to give a yellow solid residue which was crystallised from ethyl acetate:hexane give a white solid (0.278 g). This was identified as 10-desmethyl-tetrabenazine from its ¹H-NMR spectrum and ¹³C-NMR spectrum and the mass spectrum was consistent with the expected structure. HPLC analysis gave a purity of 97.9% (peak area %). HPLC-RT: 29.5 MS: M+1: 304.3

### Example 3

### 9-Desmethyl-α-dihydrotetrabenazine

A mixture of 9-desmethyltetrabenazine (1.00 g, 3.3 mmol) and sodium borohydride (0.25 g, 6.6 mmol) was stirred in a mixture of methanol:dichloromethane (1:1) (100 ml) at room temperature. TLC analysis [silica, eluting with ethyl acetate:hexane (1:1)] of an aliquot of the reaction mixture after one hour showed no starting material remained. The reaction mixture was then concentrated to dryness at reduced pressure and the residual solid partitioned between water (50 ml) and dichloromethane (50 ml). The organic layer was separated and the aqueous layer further extracted with dichloromethane (2×50 ml). The combined organic layers were dried over anhydrous potassium carbonate, filtered and concentrated at reduced pressure to give a solid which was crystallised from methanol to give a white solid (0.27 g). The ¹H-NMR spectrum and the mass spectrum were consistent with the structure of 9-desmethyl-α-dihydrotetrabenazine. HPLC analysis gave a purity of 97.3% (peak area %). HPLC-RT: 22.11 MS: M+1: 306.2

### Example 4

### 9-Desmethyl-β-dihydrotetrabenazine

9-Desmethyltetrabenazine (1.30 g, 4.28 mmol) was dissolved with stirring at 0° C. (ice bath) in dry tetrahydrofuran (30 ml) under an argon atmosphere. A solution of L-selectride (8.50 ml, 1M in THF) was added dropwise at 0° C. via a syringe to the stirring solution and the reaction mixture was then allowed to warm to room temperature. The reaction mixture was stirred for three hours and was then poured into water (40 ml) containing glacial acetic acid (4 ml). The resulting mixture was then washed with diethyl ether (2×30 ml) and was made alkaline (pH=9) by addition of solid sodium carbonate. This alkaline mixture was then extracted using dichloromethane (3×60 ml). The combined organic layers were dried over anhydrous potassium carbonate, filtered and concentrated at reduced pressure to give a solid residue which was crystallised from methanol:ethyl acetate to give a white solid (0.44 g). The ¹H-NMR spectrum and the mass spectrum were consistent with the structure of 9-desmethyl-β-dihydrotetrabenazine. HPLC analysis gave a purity of 98.2% (peak area %). HPLC-RT: 19.01 MS: M+1: 306.2

### Example 5

### 10-Desmethyl-α-dihydrotetrabenazine

10-Desmethyltetrabenazine (1.30 g, 4.3 mmol) and sodium borohydride (0.32 g, 8.6 mmol) were stirred together in a mixture of methanol:dichloromethane (1:1) (100 ml) at room temperature. TLC analysis [silica, eluting with ethyl acetate:hexane (1:1)] of an aliquot of the reaction mixture after one hour showed no starting material remained. The reaction mixture was then concentrated to dryness at reduced pressure and the residual solid partitioned between water (50 ml) and dichloromethane (50 ml). The organic layer was separated and the aqueous layer further extracted with dichloromethane (2×50 ml). The combined organic layers were dried over anhydrous potassium carbonate, filtered and concentrated at reduced pressure to give a solid which was crystallised from methanol to give a white solid (0.477 g). The ¹H-NMR spectrum and the mass spectrum were consistent with the structure of 10-Desmethyl-α-dihydrotetrabenazine. HPLC analysis gave a purity of 98.6% (peak area %). HPLC-RT: 21.28 MS: M+1: 306.3

### Example 6

### 10-Desmethyl-β-dihydrotetrabenazine

10-Desmethyltetrabenazine (1.50 g, 4.9 mmol) was dissolved with stirring at 0° C. (ice bath) in dry tetrahydrofuran (30 ml) under an argon atmosphere. A solution of L-selectride (10.00 ml, 1M in THF) was added dropwise at 0° C. via syringe to the stirring solution and the reaction mixture was then allowed to warm to room temperature. The reaction mixture was stirred for three hours and was then poured into water (40 ml) containing glacial acetic acid (4 ml). The resulting mixture was washed with diethyl ether (2×30 ml) and was made alkaline (pH=9) by the addition of solid sodium carbonate. This alkaline mixture was then extracted using dichloromethane (3×60 ml). The combined organic layers were dried over anhydrous potassium carbonate, filtered and concentrated at reduced pressure to give a solid residue which was crystallised from methanol:ethyl acetate to give a white solid (0.91 g). The ¹H-NMR spectrum and the mass spectrum were consistent with the structure of 10-Desmethyl-β-dihydrotetrabenazine. HPLC analysis gave a purity of 98.3% (peak area %). HPLC-RT: 19.84 MS: M+1: 306.3

### Example 7

Resolution of (±)-10-desmethyltetrabenazine

Resolution of the (+) and (−) isomers of 10-desmethyltetrabenazine from the corresponding racemate (see Example 2) was carried out as described below.

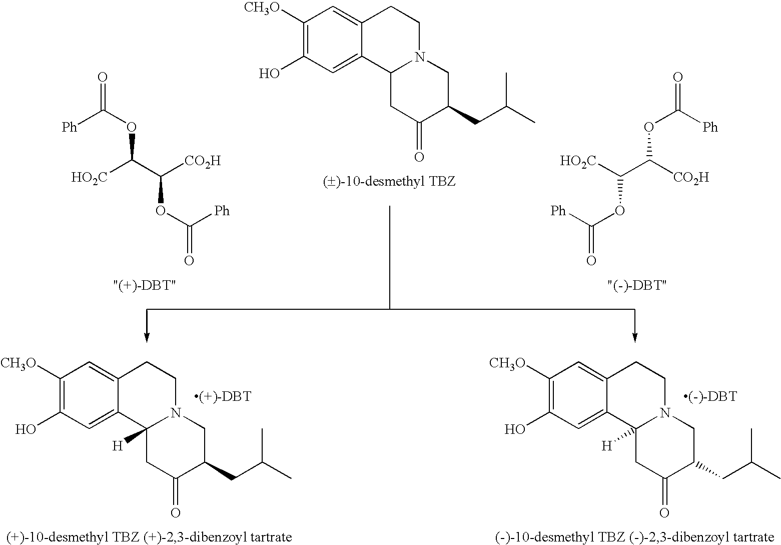

(±)-10-Desmethyltetrabenazine and (+)-2,3-dibenzoyl tartaric acid were dissolved in IDA (industrial denatured alcohol) at room temperature. On cooling, a crystalline solid formed and was collected by filtration. The solid consisted of a salt enriched in (+)-10-desmethyl tetrabenazine (+)-2,3-dibenzoyl tartrate.

The collected solid was re-crystallized from IDA to give (+)-tetrabenazine (+)-2,3-dibenzoyltartrate as a single isomer on analysis by chiral HPLC. The salt was isolated as a white solid by filtration.

(−)-Tetrabenazine (−)-2,3-dibenzoyltartrate was prepared in a similar manner using (±)-10-desmethyl tetrabenazine and (−)-2,3-dibenzoyl tartaric acid.

A sample of each enantiomer was converted to the free base and the Optical Rotation was measured (as a solution in methanol):

| Isomer | ORD |
|---|---|
| (+)-10-desmethyltetrabenazine | +64.7° |
| (−)-10-desmethyltetrabenazine | −64.1° |

### Example 8

### Synthesis of 10-desmethyl-γ-dihydrotetrabenazine isomers from racemic 10-desmethyl tetrabenazine

Following the procedure described and illustrated above in Scheme 15 and using the chlorination/dechlorination and hydroboration/oxidation procedures described in Scheme 6 (see also the examples of WO2005/077946), gave rise to a mixture of the 10-desmethyl-γ-dihydrotetrabenazine isomers (3b-VII) and (3b-VIII).

Mosher's esters of the two isomers (3b-VII) and (3b-VIII) were prepared according to the methods described in WO2005/077946, see in particular Example 1D in page 25 of WO2005/077946.

The Mosher's esters were separated by chiral chromatography and then hydrolyzed using lithium hydroxide to give the individual isomers 3b-VII) and (3b-VIII) which were converted to their mesylate salts.

In chiral HPLC analyses (using a column and conditions substantially as described in the examples of WO2005/077946), the (+) isomer eluted before the (−) isomer.

The absolute configurations of the individual isomers can be determined by X-ray crystallography following the methods described in WO2007/007105 (see in particular Example 4 on pages 42 to 49.

### Example 9

### Synthesis of (−)-10-desmethyl-γ-dihydrotetrabenazine from (−)-10-desmethyl tetrabenazine

(−)-10-Desmethyl-γ-dihydrotetrabenazine was prepared from (−)-10-desmethyl tetrabenazine by following the sequence of reactions described in Scheme 14 above. Measurement of the optical rotation of the (−)-10-desmethyl-γ-dihydrotetrabenazine gave a value of −78°. Analysis by chiral HPLC showed co-elution of the product with the 10-desmethyl-γ-dihydrotetrabenazine isomer that eluted second in the HPLC analysis of the mixture of isomers obtained from the synthesis described in Example 8.

### Biological Activity

### Example 10

### Screen for VMAT-2 binding activity using a [³H] Dihydrotetrabenazine binding Assay

Dihydrotetrabenazine is a very potent and selective inhibitor of VMAT-2, and binds with high affinity (nM range) to this vesicular transporter. [³H] Dihydrotetrabenazine has been successfully used for many years as a radioligand to label VMAT-2 in human, bovine and rodent brain (e.g. Scherman et al. J. Neurochem. 50, 1131-1136 (1988); Near et al. Mol. Pharmacol. 30, 252-257 (1986); Kilbourn et al. Eur. J. Pharmacol. 278, 249-252 (1995); and Zucker et al. Life Sci. 69, 2311-2317 (2001)).

The compounds of the invention can be tested for their ability to inhibit the VMAT-2 transporter using the assay described below.

### Assay A

Adult rat (Wistar strain) forebrain membranes are prepared essentially as described by Chazot et al. (1993) Biochem. Pharmacol. 45, 605-610. Adult rat striatal vesicular membranes are prepared essentially as described by Roland et al. (2000), JPET 293, 329-335. 10 μg Membranes are incubated at 25° C. with [³H] dihydrotetrabenazine (18-20 nM) in 50 mM HEPES pH 8.0 (assay buffer), for 60 minutes, and bound radioligand is collected by rapid filtration under vacuum on GF/B glass-fibre filters. Non-specific binding is determined in parallel samples in the presence of 2 μM unlabelled tetrabenazine. Radioactivity is counted in scintillation fluid in a β-counter. A full concentration range (log and half-log units) of the test compounds is assayed (range: 10⁻¹¹-10⁻⁴M) in triplicate. Test compounds and tetrabenazine are dissolved in DMSO at a stock concentration of 10 mM, and dilutions then prepared in assay buffer. Three independent experiments are performed for each compound. Data are analysed and curve fitted using the GraphPad Prism 3.2 package.

### Assay B

Charles River CD-1 rats were euthanized by decapitation and the brains dissected free and placed in ice cold PBS. The chilled brain was placed in a coronal brain matrix (Activational Systems, Inc) with 1 mm intervals. Single edge razor blades were inserted such that a 6 mm slice incorporating the striatum was removed. The tissue was weighed and placed in an ice-cold hand homogenizer, 10 volumes of KBS-EDTA (137 mM KCl, 3 mM NaCl, 8 mM K₂HPO₄, 1.5 mM NaH₂PO₄, 1 mM EDTA, pH 8.0) were added and the tissue homogenized with 10 strokes (maintaining on ice). Aliquots were placed into 500 μl microfuge tubes and then stored at −80° C. for later assays. A fresh aliquot was taken for protein assay. Protein concentrations were determined using BCA Protein Assay (Pierce Chemical) with bovine serum albumin as the standard. For VMAT2 assay, aliquots were thawed and placed on ice, and diluted with KBS-EDTA for a final protein concentration of 1 mg/ml.

Whatman GF/C filters (Acros) were presoaked with PBS/0.1% polyethylenimine at room temperature and then placed on a 48 place cell harvester (Brandel). Harvester was loaded with labeled tubes (12×75 mm) in the test tube rack. Into the tubes were placed 10 μl of the compound dilutions to their respective triplicate tubes and 1 μl of 10 mM TBZ added to the tubes used for non-specific binding measurements. Into each tube was then placed 0.5 ml of a 10 nM [³H] dihydrotetrabenazine (DTBZ) dilution (American Radiolabeled Chemicals) in KBS-EDTA, the tubes were vortexed, and then 0.5 ml of the 1 mg/ml rat forebrain tissue homogenate solution was added into all tubes. Each tube was vortexed briefly and then incubated at room temperature for 30 minute.

Final concentrations of samples were 500 μg protein, 5 nM [3H]DTBZ; 10 μM TBZ (for nonspecific tubes) in 1 ml total volume. The samples were run through the cell harvester followed by 2 washes of 2 ml KBS-EDTA. Filter circles were placed in scintillation vials and 10 ml of Universol (ICN) was added, samples incubated overnight and then counted on a Packard Tri-Carb 1600 scintillation counter.

Competition binding isotherms were analyzed with the use of computer-assisted nonlinear curve-fitting (LIGAND program) using both one and two site models.

The following binding affinities were determined:

| Compound | Kᵢ (nM) | Bₘₐₓ (fmol/μg) |
|---|---|---|
| (−)-10-desmethyl-γ- | 1.82 +/− 0.47 | 0.101 +/− 0.009 |
| dihydrotetrabenazine | | |
| (+)-10-desmethyl-γ- | 450.7 +/− 141.7 | 0.174 +/− 0.035 |
| dihydrotetrabenazine | | |

### Example 11

### VMAT Functional Assays

### A. VMAT2 Functional Assay

Rat striatal synaptic vesicles are prepared essentially as described in Example 10. Thus, a rat striatal P₂ membrane preparation (Chazot et al., 1993) is resuspended and homogenized in ice-cold distilled water. Osmolarity is restored by addition of 25 mM HEPES and 100 mM potassium tartrate (pH 7.5, 4C). The preparation is then centrifuged for 20 minutes at 20,000×g (4° C.). The resultant S₃ fraction is removed, magnesium sulphate is added (to give a final concentration of 1 mM, pH 7.5, 4° C.), and the mixture is centrifuged at 100,000×g for 45 minutes. The final P₄ fraction contains the synaptic vesicles for the assay.

An aliquot of 100 μl (approx. 2.5 μg protein) of synaptic vesicles is preincubated with increasing concentrations of the test compounds (prepared fresh as a stock of 10⁻²M in DMSO) for 30 minutes (concentration range 10⁻⁹ M-10⁻⁴ M), and then for 3 minutes in the assay buffer (25 mM HEPES, 100 mM potassium tartrate, 1.7 mM ascorbic acid, 0.05 mM EGTA, 0.1 mM EDTA, 2 mM ATP—Mg²⁺, pH 7.5), in the presence of [³H] dopamine (30 nM final concentration) at 30° C. The reaction is then terminated by addition of ice-cold buffer assay buffer pH 7.5, containing 2 mM MgSO₄ instead of 2 mM ATP—Mg²⁺, and rapid filtration achieved through Whatman filters soaked in 0.5% polyethyleneimine. The filters are washed three times with cold buffer using a Brandel Harvester. The radioactivity trapped on the filters is counted using a liquid scintillation counter and non-specific binding is determined by measuring vesicular [³H] dopamine uptake at 4° C. The method is based on that described in Ugarte Y V et al. (2003) Eur. J. Pharmacol. 472, 165-171. Selective VMAT-2 uptake is defined using 10 μM tetrabenazine.

### B. VMAT1 Functional Assay

There are very limited native tissues which possess VMAT1 alone, in isolation from VMAT2. However, tetrabenazine displays at least a 200-fold higher affinity for VMAT2 in comparison to VMAT1, and this discrimination can be used to block the influence of VMAT2 in the functional assay (Erickson et al. (1996) PNAS (USA) 93, 5166-5171).

Adrenal chromaffin cells are isolated from young adult SD rats essentially as described in Moshharov et al. (2003) J Neurosci. 23, 5835-5845. Thus, adrenal glands are dissected in ice cold PBS, the capsule and cortex of the glands removed and the remaining medullae are minced. After multiple washes with PBS, the tissue is incubated with Ca2+-free collagenase IA solution (250 U/ml) for 30 minutes at 30° C. with gentle stirring. The digested tissue is rinsed three times and the dissociated cells are centrifuged at 3000 rpm to form a pellet, which is resuspended in PBS. The vesicular fraction is isolated in an identical fashion to that described for the brain preparation.

100 μl (approx. 2.5 μg protein) of synaptic vesicles are preincubated with increasing concentrations of test compound (prepared as previously described for binding assay) for 30 minutes (concentration range 10⁻⁹ M-10⁻⁴ M). The assay is performed for 3 minutes at 30° C. in the assay buffer (25 mM HEPES, 100 mM potassium tartrate, 1.7 mM ascorbic acid, 0.05 mM EGTA, 0.1 mM EDTA, 2 mM ATP—Mg²⁺, pH 7.5), in the presence of [³H] dopamine (30 nM final concentration). [³H] dopamine uptake is measured in the presence of 10 μM tetrabenazine (selectively blocks VMAT2 at this concentration). Non-specific uptake is determined by measuring vesicular [³H] dopamine uptake at 4° C. The reaction is then terminated by addition of ice-cold buffer assay buffer pH 7.5, containing 2 mM MgSO₄ instead of 2 mM ATP—Mg²⁺, and rapid filtration achieved through Whatman filters soaked in 0.5% polyethyleneimine. The filters are washed three times with cold buffer using a Brandel Harvester and the radioactivity trapped on the filters is counted using a liquid scintillation counter.

### Example 12

### Receptor and Transporter Protein Binding Studies

Compounds of the invention were subjected to specific binding assays to test their ability to bind to the receptors and transporter proteins described below. The results are shown in the table.

### (a) Adrenergic α_{2A} Receptor:

Reference: S. Uhlçn et al. J. Pharmacol. Exp. Ther., 271:1558-1565 (1994)
Source: Human recombinant insect Sf9 cells
Ligand: 1 nM [³H] MK-912
Vehicle: 1% DMSO
Incubation time/Temp: 60 minutes @ 25° C.
Incubation buffer: 75 mM Tris-HCl, pH 7.4, 12.5 mM MgCl₂, 2 mM EDTA
Non Specific ligand: 10 μM WB-4101
K_{d}: 0.6 nM
Bₘₐₓ: 4.6 pmole/mg protein
Specific binding: 95%
Quantitation method: Radioligand binding
Significance criteria: ≧50% of maximum stimulation or inhibition

### (b) Adrenergic α_{2B} Receptor:

Reference: S. Uhlen et al., Eur. J. Pharmacol., 343 (1): 93-1-01 (1998)
Source: Human recombinant CHO-K1 cells
Ligand: 2.5 nM [3H] Rauwolscine
Vehicle: 1% DMSO
Incubation time/Temp: 60 minutes @ 25° C.
Incubation buffer: 50 mM Tris-HCl, 1 mM EDTA, 12.5 mM MgCl₂, pH 7.4, 0.2% BSA at 25° C.
Non Specific ligand: 10 μM Prazosin
K_{d}: 2.1 nM
Bₘₐₓ: 2.1 pmole/mg protein
Specific binding: 90%
Quantitation method: Radioligand binding
Significance criteria: ≧50% of maximum stimulation or inhibition

### (c) Adrenergic α_{2C} Receptor:

Reference: S. Uhlçn et al. J. Pharmacol. Exp. Ther., 271:1558-1565 (1994)
Source: Human recombinant insect Sf9 cells
Ligand: 1 nM [³H] MK-912
Vehicle: 1% DMSO
Incubation time/Temp: 60 minutes @ 25° C.
Incubation buffer: 75 mM Tris-HCl, pH 7.4, 12.5 mM MgCl₂, 2 mM EDTA
Non Specific ligand: 10 μM WB-4101
K_{D}: 0.17 nM
Bₘₐₓ: 6.8 pmole/mg protein
Specific binding: 95%
Quantitation method: Radioligand binding
Significance criteria: ≧50% of maximum stimulation or inhibition

### (d) Dopamine D₁ Receptor:

Reference: Dearry et al., Nature, 347:72-76, (1990)
Source: Human recombinant CHO cells
Ligand: 1.4 nM [3H] SCH-23390
Vehicle: 1% DMSO
Incubation time/Temp: 2 hours @ 37° C.
Incubation buffer: 50 mM Tris-HCl, pH 7.4, 150 mM NaCl, 1.4 mM ascorbic acid, 0.001% BSA
Non Specific ligand: 10 μM (+)-butaclamol
K_{d}: 1.4 nM
Bₘₐₓ: 0.63 pmole/mg protein
Specific binding: 90%
Quantitation method: Radioligand binding
Significance criteria: ≧50% of maximum stimulation or inhibition

### (e) Dopamine D_{2L} Receptor:

Reference: Grandy et al., Proc. Natl. Acad. Sci. USA, 86:9762-9766 (1989)
Source: Human recombinant CHO cells
Ligand: 0.16 nM [³H] Spiperone
Vehicle: 1% DMSO
Incubation time/Temp: 2 hours @ 25° C.
Incubation buffer: 50 mM Tris-HCl, pH 7.4, 150 mM NaCl, 1.4 mM ascorbic acid, 0.001% BSA
Non Specific ligand: 10 μM Haloperidol
K_{d}: 0.08 nM
Bₘₐₓ: 0.48 pmole/mg protein
Specific binding: 85%
Quantitation method: Radioligand binding
Significance criteria: ≧50% of maximum stimulation or inhibition

### (f) Dopamine D_{2S} Receptor:

Reference: Grandy et al., Proc. Natl. Acad. Sci. USA, 86:9762-9766 (1989)
Source: Human recombinant CHO cells
Ligand: 0.16 nM [³H] Spiperone
Vehicle: 1% DMSO
Incubation time/Temp: 2 hours @ 25° C.
Incubation buffer: 50 mM Tris-HCl, pH 7.4, 150 mM NaCl, 1.4 mM ascorbic acid, 0.001% BSA
Non Specific ligand: 10 μM Haloperidol
K_{d}: 0.09 nM
Bₘₐₓ: 1.6 pmole/mg protein
Specific binding: 90%
Quantitation method: Radioligand binding
Significance criteria: ≧50% of maximum stimulation or inhibition

### (g) Dopamine D₃ Receptor:

Reference: Sokoloff et al., Nature, 347:146-151, (1990)
Source: Human recombinant CHO cells
Ligand: 0.7 nM [3H] Spiperone
Vehicle: 1% DMSO
Incubation time/Temp: 2 hours @ 37° C.
Incubation buffer: 50 mM Tris-HCl, pH 7.4, 150 mM NaCl, 1.4 mM ascorbic acid, 0.001% BSA
Non Specific ligand: 25 μM S(−)-Sulpiride
K_{d}: 0.36 nM
Bₘₐₓ: 1.1 pmole/mg protein
Specific binding: 85%
Quantitation method: Radioligand binding
Significance criteria: ≧50% of maximum stimulation or inhibition

### (h) Dopamine D_{4.2} Receptor:

Reference: Van Tol et al., Nature, 350:610-614 (1991)
Source: Human recombinant CHO-K1 cells
Ligand: 0.5 nM [³H] Spiperone
Vehicle: 1% DMSO
Incubation time/Temp: 2 hours @ 25° C.
Incubation buffer: 50 mM Tris-HCl, pH 7.4, 150 mM NaCl, 1.4 mM ascorbic acid, 0.001% BSA
Non Specific ligand: 10 μM Haloperidol
K_{d}: 0.32 nM
Bₘₐₓ: 0.55 pmole/mg protein
Specific binding: 90%
Quantitation method: Radioligand binding
Significance criteria: ≧50% of maximum stimulation or inhibition

### (i) Dopamine D_{4.4} Receptor:

Reference: Van Tol et al., Nature, 350:610-614 (1991)
Source: Human recombinant CHO-K1 cells
Ligand: 1.2 nM [³H] Spiperone
Vehicle: 1% DMSO
Incubation time/Temp: 2 hours @ 25° C.
Incubation buffer: 50 mM Tris-HCl, pH 7.4, 150 mM NaCl, 1.4 mM ascorbic acid, 0.001% BSA
Non Specific ligand: 10 μM Haloperidol
K_{d}: 0.46 nM
Bₘₐₓ: 0.63 pmole/mg protein
Specific binding: 85%
Quantitation method: Radioligand binding
Significance criteria: ≧50% of maximum stimulation or inhibition

### (j) Dopamine D_{4.7} Receptor:

Reference: Van Tol et al., Nature, 350:610-614 (1991)
Source: Human recombinant CHO-K1 cells
Ligand: 1.5 nM [³H] Spiperone
Vehicle: 1% DMSO
Incubation time/Temp: 2 hours @ 25° C.
Incubation buffer: 50 mM Tris-HCl, pH 7.4, 150 mM NaCl, 1.4 mM ascorbic acid, 0.001% BSA
Non Specific ligand: 10 μM Haloperidol
K_{d}: 0.48 nM
Bₘₐₓ: 0.77 pmole/mg protein
Specific binding: 85%
Quantitation method: Radioligand binding
Significance criteria: ≧50% of maximum stimulation or inhibition

### (k) Imidazoline I₂ (Central) Receptor:

Reference: Brown et al., Brit. J. Pharmacol., 99:803-809, (1990)
Source: Wistar rat cerebral cortex
Ligand: 2 nM [³H] Idazoxan
Vehicle: 1% DMSO
Incubation time/Temp: 30 minutes @ 25° C.
Incubation buffer: 50 mM Tris-HCl, 0.5 mM EDTA, pH 7.4 at 25° C.
Non Specific ligand: 1 μM Idazoxan
K_{d}: 4 nM
Bₘₐₓ: 0.14 pmole/mg protein
Specific binding: 85%
Quantitation method: Radioligand binding
Significance criteria: ≧50% of maximum stimulation or inhibition

### (I) Imidazoline I₂ (Peripheral) Receptor:

Reference: Brown et al., Brit. J. Pharmacol., 99:803-809, (1990)
Source: Wistar rat kidney
Ligand: 5 nM [³H] Idazoxan
Vehicle: 1% DMSO
Incubation time/Temp: 90 minutes @ 25° C.
Incubation buffer: 50 mM Tris-HCl, 0.5 mM EDTA, pH 7.4 at 25° C.
Non Specific ligand: 1 μM Idazoxan
K_{d}: 0.011 nM
Bₘₐₓ: 0.069 pmole/mg protein
Specific binding: 70%
Quantitation method: Radioligand binding
Significance criteria: ≧50% of maximum stimulation or inhibition

### (m) Sigma σ₁ Receptor:

Reference: Ganaphthy et al., Pharmacol. Exp. Ther., 289:251-260, (1999)
Source: Human jurkat cells
Ligand: 8 nM [3H] Haloperidol
Vehicle: 1% DMSO
Incubation time/Temp: 4 hours @ 25° C.
Incubation buffer: 5 mM K2HPO4/KH2PO4 buffer pH 7.5
Non Specific ligand: 10 μM Haloperidol
K_{d}: 5.8 nM
Bₘₐₓ: 0.71 pmole/mg protein
Specific binding: 80%
Quantitation method: Radioligand binding
Significance criteria: ≧50% of maximum stimulation or inhibition

### (n) Sigma σ₂ Receptor:

Reference: Hashimoto et al., Eur. J. Pharmacol., 236:159-163, (1993)
Source: Wistar rat brain
Ligand: 3 nM [3H] Ifenprodil
Vehicle: 1% DMSO
Incubation time/Temp: 60 minutes @ 37° C.
Incubation buffer: 50 mM Tris-HCl, pH 7.4
Non Specific ligand: 10 μM Ifenprodil
K_{d}: 4.8 nM
Bₘₐₓ: 1.3 pmole/mg protein
Specific binding: 85%
Quantitation method: Radioligand binding
Significance criteria: ≧50% of maximum stimulation or inhibition

### (o) Serotonin Transporter (SERT):

Reference: Gu et al., J. Biol. Chem., 269(10):7124-7130, (1994)
Source: Human recombinant HEK-293 cells
Ligand: 0.15 nM [125I] RTI-55
Vehicle: 1% DMSO
Incubation time/Temp: 3 hours @ 4° C.
Incubation buffer: 100 mM NaCl, 50 mM Tris HCl, 1 μM Leupeptin, 10 μM PMSF, pH 7.4
Non Specific ligand: 10 μM Imipramine
K_{d}: 0.17 nM
Bₘₐₓ: 0.41 pmole/mg protein
Specific binding: 95%
Quantitation method: Radioligand binding
Significance criteria: ≧50% of maximum stimulation or inhibition

### (p) Dopamine Transporter (DAT):

Reference: Giros et al., Trends Pharmacol. Sci., 14, 43-49 (1993) Gu et al., J. Biol. Chem., 269(10):7124-7130 (1994)
Source: Human recombinant CHO cells
Ligand: 0.15 nM [¹²⁵I] RTI-55
Vehicle: 1% DMSO
Incubation time/Temp: 3 hours @ 4° C.
Incubation buffer: 100 mM NaCl, 50 mM Tris HCl, 1 μM Leupeptin, 10 μM PMSF, pH 7.4 Non Specific ligand: 10 μM Nomifensine
K_{d}: 0.58 nM
Bₘₐₓ: 0.047 pmole/mg protein
Specific binding: 90%
Quantitation method: Radioligand binding
Significance criteria: ≧50% of maximum stimulation or inhibition

The results of the receptor and enzyme binding assays of (+)-10-desmethyl-γ-dihydrotetrabenazine and (−)-10-desmethyl-γ-dihydrotetrabenazine (see Examples 8 and 9) are set out in the table below. The figures given are the % inhibition values for the compounds at a concentration of 10 μM.

| | (+)-10-desmethyl-γ- | (−)-10-desmethyl-γ- |
|---|---|---|
| Receptor/Protein | dihydrotetrabenazine | dihydrotetrabenazine |
| (a) α_{2A} Receptor | 13 | 69 |
| (b) α_{2B} Receptor | 21 | 44 |
| (c) α_{2C} Receptor | −7 | 60 |
| (d) D₁ Receptor | 29 | 96 |
| (e) D_{2L} Receptor | 54 | 98 |
| (f) D_{2S} Receptor | 63 | 98 |
| (g) D₃ Receptor | 65 | 101 |
| (h) D_{4.2} Receptor | 55 | 90 |
| (i) D_{4.4} Receptor | 33 | 81 |
| (j) D_{4.7} Receptor | 25 | 87 |
| (k) I₂ Receptor | 24 | 66 |
| (central) | | |
| (l) I₂ Receptor | 24 | 25 |
| (peripheral) | | |
| (m) σ₁ Receptor | 83 | 55 |
| (n) σ₂ Receptor | 31 | 32 |

The results indicate that (−)-10-desmethyl-γ-dihydrotetrabenazine is a potent inhibitor of dopamine receptors.

### Example 13

### Enzyme Assays

Compounds of the invention can be tested for their ability to inhibit enzymes involved in the processing of monoamines in the CNS, namely Catechol O-Methyl Transferase (COMT), Monoamine Oxidase A and Monoamine Oxidase B.

### (a) Catechol O-Methyl Transferase (COMT) Inhibition

Source: Porcine liver
Substrate: 3 mM catechol +S-adenosyl-L-[³H]methionine
Vehicle: 1% DMSO
Pre-incubation time/Temp: None
Incubation time: 60 minutes @ 37° C.
Incubation buffer: 100 mM potassium phosphate, 10 mM MgCl₂, 3 mM DTT containing 12 units/ml adenosine deaminase, pH 7.4
Quantitation method: Quantitation of [³H] guiacol.
Significance criteria: ≧50% of maximum stimulation or inhibition

### (b) Monoamine Oxidase MAO-A Inhibition

Source: Human recombinant
Substrate: 50 μM kynuramine
Vehicle: 1% DMSO
Pre-incubation time/Temp: 15 minutes @ 37° C.
Incubation time: 60 minutes @ 37° C.
Incubation buffer: 100 mM KH₂PO₄, pH 7.4
Quantitation method: Spectrofluorimetric quantification of 4-hydroxyquinoline
Significance criteria: ≧50% of maximum stimulation or inhibition

### (c) Monoamine Oxidase MAO-B Inhibition

Source: Human recombinant
Substrate: 50 μM kynuramine
Vehicle: 1% DMSO
Pre-incubation time/Temp: 15 minutes @ 37° C.
Incubation time: 60 minutes @ 37° C.
Incubation buffer: 100 mM KH₂PO₄, pH 7.4
Quantitation method: Spectrofluorimetric quantification of 4-hydroxyquinoline
Significance criteria: ≧50% of maximum stimulation or inhibition

### Example 14

### Cellular Assays

The ability of compounds of the invention to inhibit uptake of serotonin (5-hydroxytryptamine) by human embryonic kidney cells can be measured using the following assay conditions:
Target: Human HEK-293 cells
Vehicle: 0.4% DMSO
Incubation Time/Temp: 10 minutes @ 25° C.
Incubation buffer: 5 mM Tris-HCl, 7.5 mM HEPES, 120 mM NaCl, 5.4 mM KCl, 1.2 mM CaCl₂, 1.2 mM MgSO₄, 5 mM glucose, 1 mM ascorbic acid, pH 7.1
Quantitation Method Quantitation of [³H] serotonin uptake
Significance criteria: ≧50% inhibition of [³H] serotonin uptake relative to fluxetine response.

### Example 15

### 5-HT_{1D/1B} Binding Assay

The ability of the compounds of the invention to bind to 5-HT_{1D/1B} receptors can be tested using an assay based on the one described by Millan, M J et al. (2002) Pharmacol. Biochem. Behav. 71, 589-598. [N-methyl ³H] GR-125743 is used as the radioliogand for both 5-HT_{1D} and 5-HT_{1B} receptors. Adult SD rat forebrain P₂ membranes (Chazot et al., 1993) are used for the assay. An assay buffer such as 50 mM Tris-HCl pH 7.7 at room temperature containing 4 mM calcium chloride, 0.1% ascorbic acid and 10 μM pargyline. 5-HT (10 μM) can be used to define non-specific binding. Incubation with 1 nM [³H] GR-125743 can be carried out for 1 hour at room temperature, and the reaction is terminated by rapid filtration using a Brandel Harvester through GF/B filters pre-soaked in 0.1% polyethyleneimine, followed by three washes with ice-cold buffer (supplemented with 0.1% BSA). A dose range of 10⁻¹⁰-10⁻⁴M is utilized. The resultant competition curves are analysed using the GraphPad Prism 4 package.

### Example 16

### Pharmaceutical Compositions

### (i) Tablet Formulation-I

A tablet composition containing a compound of the invention is prepared by mixing 50 mg of the dihydrotetrabenazine with 197 mg of lactose (BP) as diluent, and 3 mg magnesium stearate as a lubricant and compressing to form a tablet in known manner.

### (ii) Tablet Formulation-II

A tablet composition containing a compound of the invention is prepared by mixing the compound (25 mg) with iron oxide, lactose, magnesium stearate, starch maize white and talc, and compressing to form a tablet in known manner.

(iii) Capsule Formulation

A capsule formulation is prepared by mixing 100 mg of a compound of the invention with 100 mg lactose and filling the resulting mixture into standard opaque hard gelatin capsules.

### EQUIVALENTS

It will readily be apparent that numerous modifications and alterations may be made to the specific embodiments of the invention described above without departing from the principles underlying the invention. All such modifications and alterations are intended to be embraced by this application.

What is claimed:

1. A compound of the formula (1): 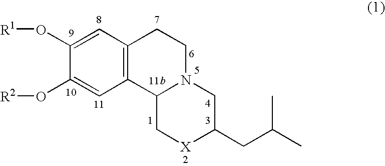
wherein R¹ and R² are each selected from hydrogen and methyl; and
X is CHOH or C═O; or
a pharmaceutically acceptable salt or tautomer thereof,
but excluding compounds 2R,3R,11bR-9-O-desmethyl-dihydrotetrabenazine and 2S,3S,11bS-9-O-desmethyl-dihydrotetrabenazine and their tautomers.

2. The compound of claim 1 having formula (1a): 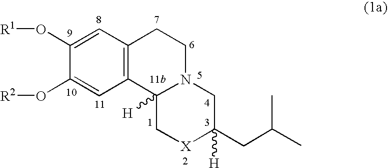
or a pharmaceutically acceptable salt or tautomer thereof, wherein R¹ and R² are each selected from hydrogen and methyl, provided that at least one of R¹ and R² is hydrogen; and X is CHOH or C═O; and
provided that the hydrogen atoms in positions 3 and 11b are present in a cis relative orientation when (i) R¹ and R² are both hydrogen, or (ii) R¹ is hydrogen and R² is methyl.

3. The compound of claim 1 wherein the hydrogen atoms in positions 3 and 11b are present in a cis orientation relative to each other.

4. The compound of claim 1 having the formula (2): 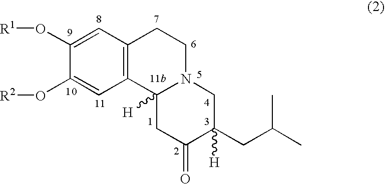
and pharmaceutically acceptable salts thereof.

5. The compound of claim 4, wherein the compound of formula (2) has the following structure:

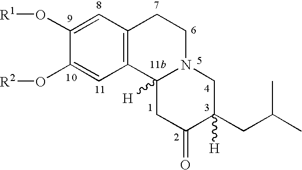

6. The compound of claim 4 having the formula (2a): 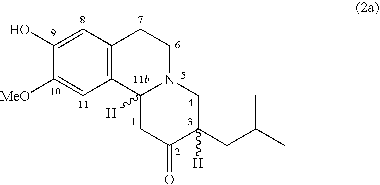
and pharmaceutically acceptable salts thereof.

7. The compound of claim 2 having the formula (2b): 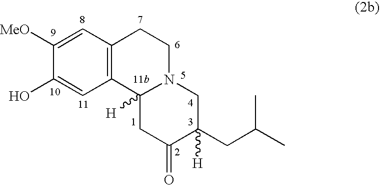
and pharmaceutically acceptable salts thereof.

8. The compound of claim 7 which is selected from compounds (2b-I) and (2b-II): 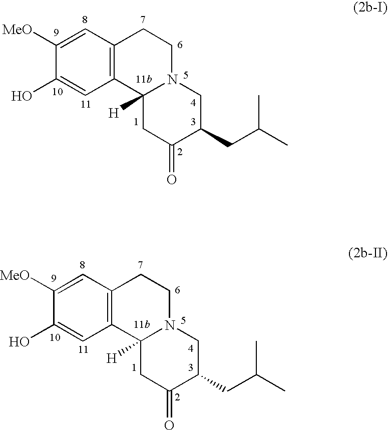
and pharmaceutically acceptable salts thereof.

9. The compound of claim 4 having the formula (2c): 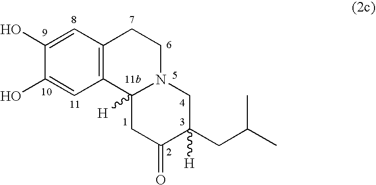
and pharmaceutically acceptable salts thereof.

10. The compound of claim 2 having the formula (3): 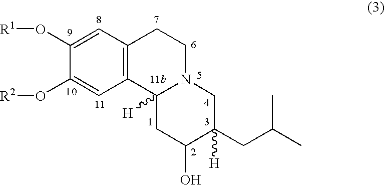
and pharmaceutically acceptable salts thereof.

11. The compound of claim 10, wherein formula (3) excludes compounds 2R,3R,11bR-9-O-desmethyl-dihydrotetrabenazine and 2S,3S,11bS-9-O-desmethyl-dihydrotetrabenazine and their salts and tautomers.

12. The compound of claim 10 having the formula (3a): 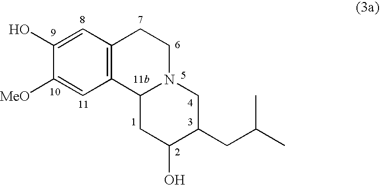
and pharmaceutically acceptable salts thereof.

13. The compound of claim 10 which is selected from compounds (3a-V), (3a-VI), (3a-VII) and (3a-VIII): 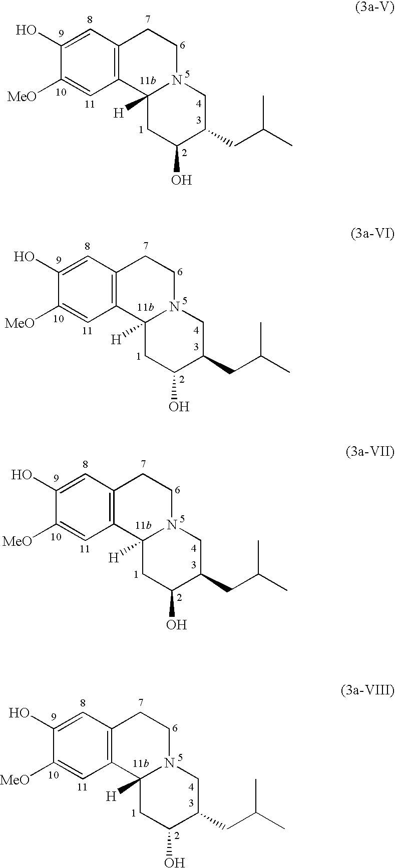
and pharmaceutically acceptable salts thereof.

14. The compound of claim 10 having the formula (3b): 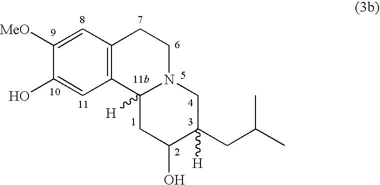
and pharmaceutically acceptable salts thereof.

15. The compound of claim 14, which is selected from compounds (3b-I), (3b-II), (3b-III) and (3b-IV): 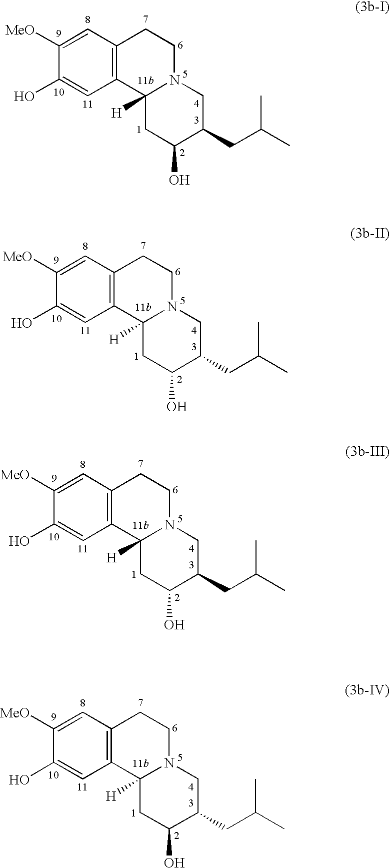
and pharmaceutically acceptable salts thereof.

16. The compound of claim 14, which is selected from the compounds (3b-V), (3b-VI) (3b-VII) and (3b-VIII): 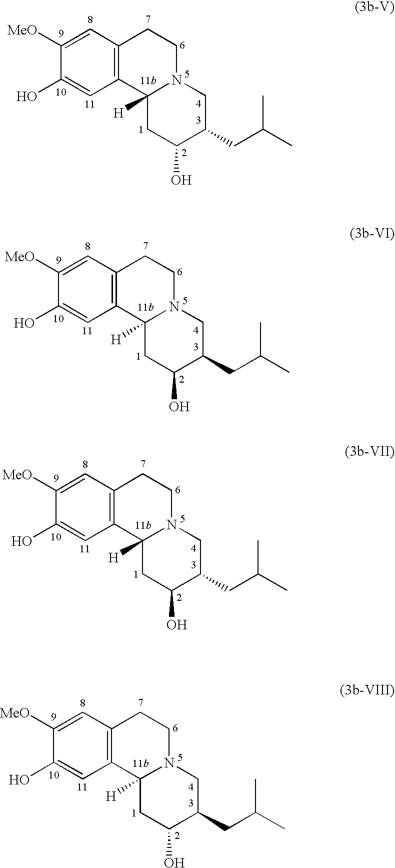
and pharmaceutically acceptable salts thereof.

17. The compound of claim 10 having the formula (3c): 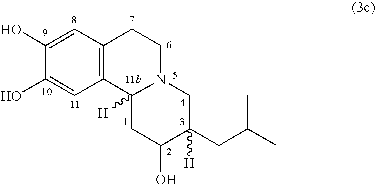
and pharmaceutically acceptable salts thereof.

18. The compound of claim 17, which is selected from compounds (3c-V), (3c-VI) (3c-VII) and (3c-VIII): 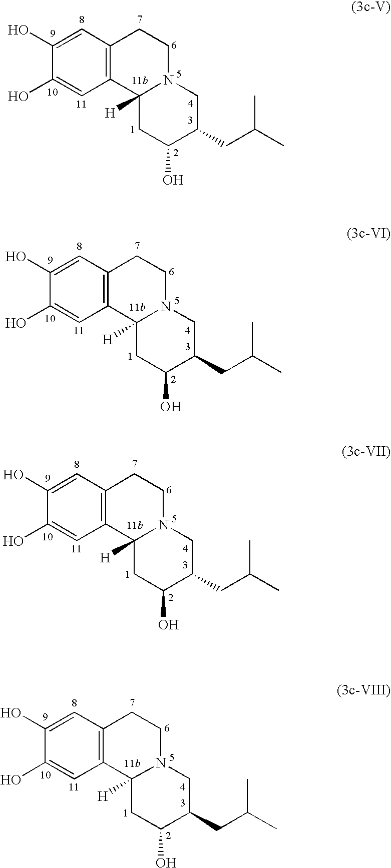
and pharmaceutically acceptable salts thereof.

19. The compound of claim 2 which is (−)-10-desmethyl-γ-dihydrotetrabenazine or a pharmaceutically acceptable salt thereof.

20. The compound of claim 2 which is (+)-10-desmethyl-γ-dihydrotetrabenazine or a pharmaceutically acceptable salt thereof.

21. The compound of the formula (1) as defined in claim 1 wherein said compound is selected from the compounds (2a-I) and (2a-II): 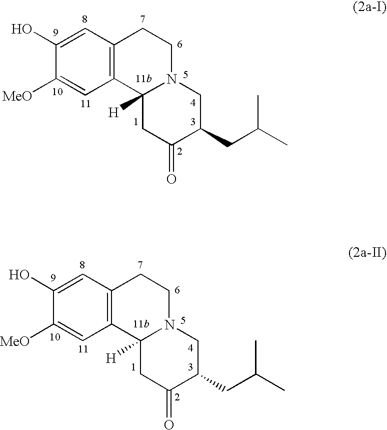
and pharmaceutically acceptable salts thereof; and the compounds (2c-I) and (2c-II): 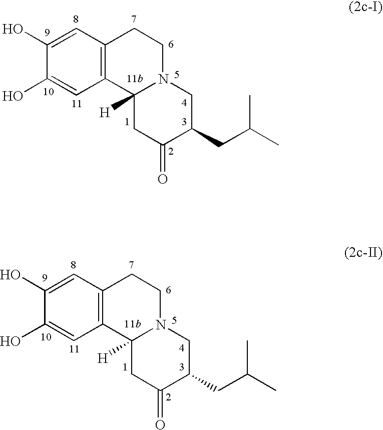
and pharmaceutically acceptable salts thereof; and the compounds (3a-I), (3a-II), (3a-III) and (3-a-IV): 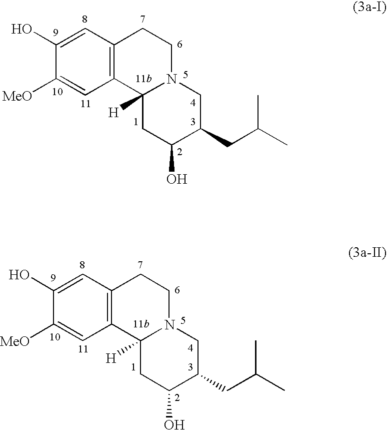
and pharmaceutically acceptable salts thereof; and the compounds (3c-I), (3c-II), (3c-III), (3c-IV): 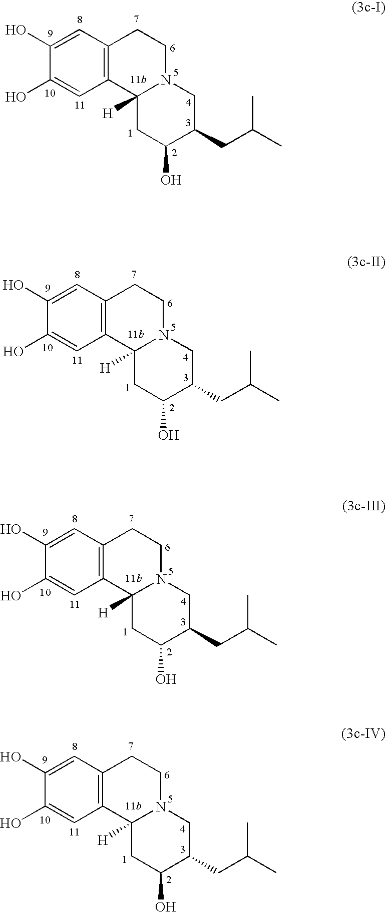
and pharmaceutically acceptable salts thereof.

22. The compound of claim 1 in the form of a pharmaceutically acceptable salt.

23. A pharmaceutical composition comprising a compound of the formula (1): 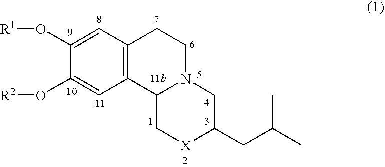
or a pharmaceutically acceptable salt or tautomer thereof, and combinations thereof;
wherein R¹ and R² are each selected from hydrogen and methyl; and
X is CHOH or C═O.

24. The composition of claim 23, wherein the composition comprises a therapeutically effective amount of the compound of the formula (1).

25. The composition of claim 23, wherein the compound is selected from the following group of compounds: 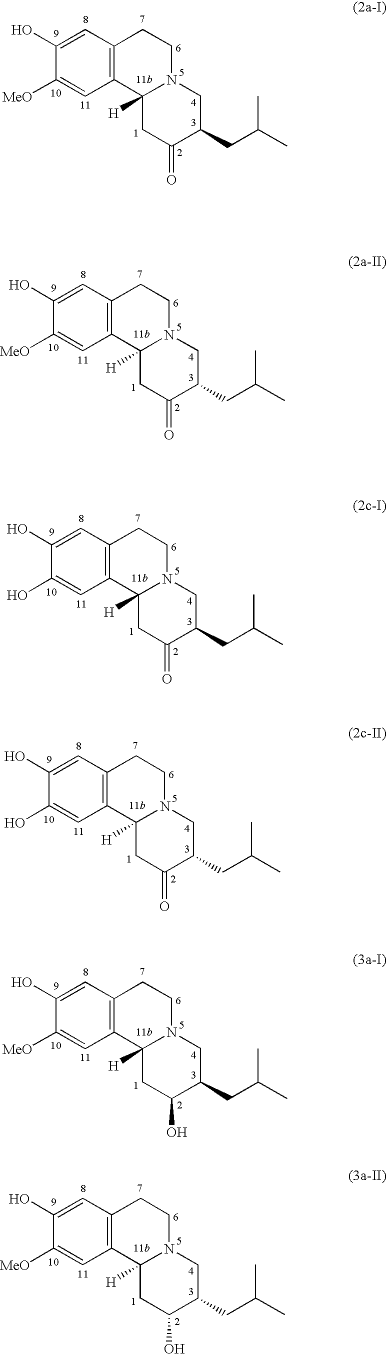 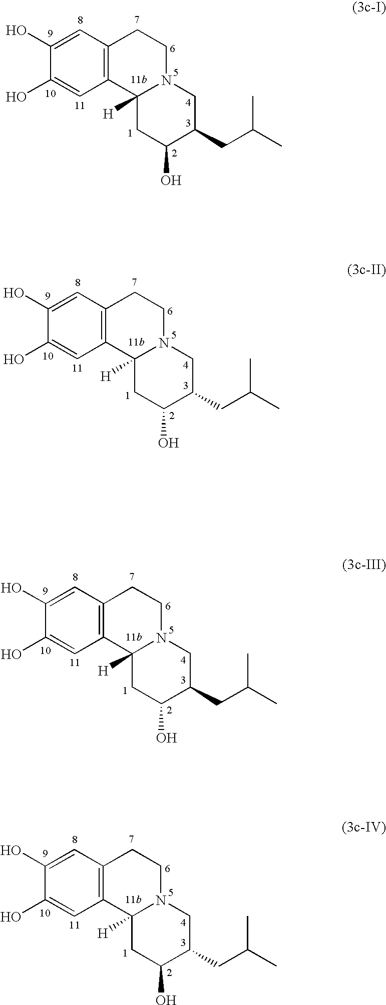
or a combination thereof, and pharmaceutically acceptable salts thereof.

26. The composition of claim 23, wherein the compound is selected from: 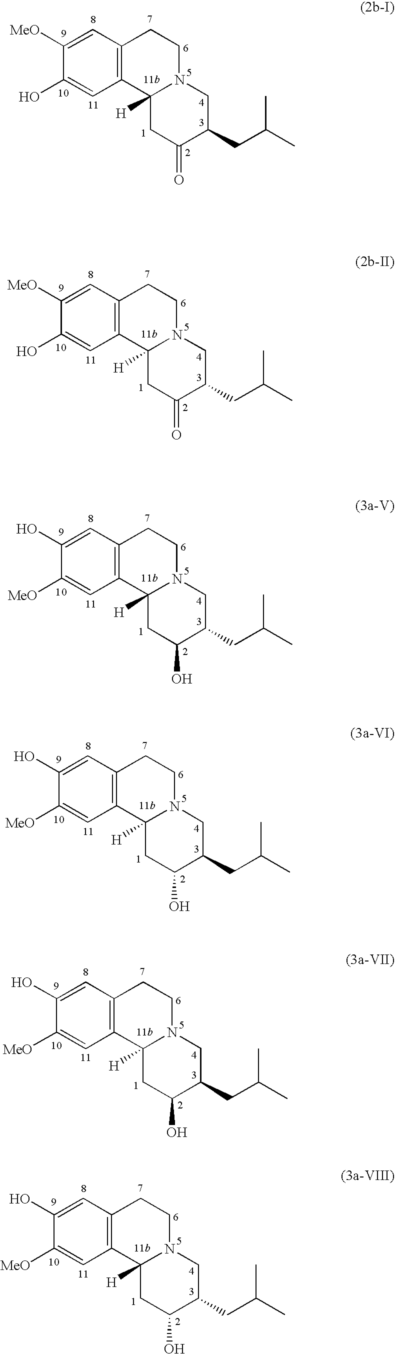 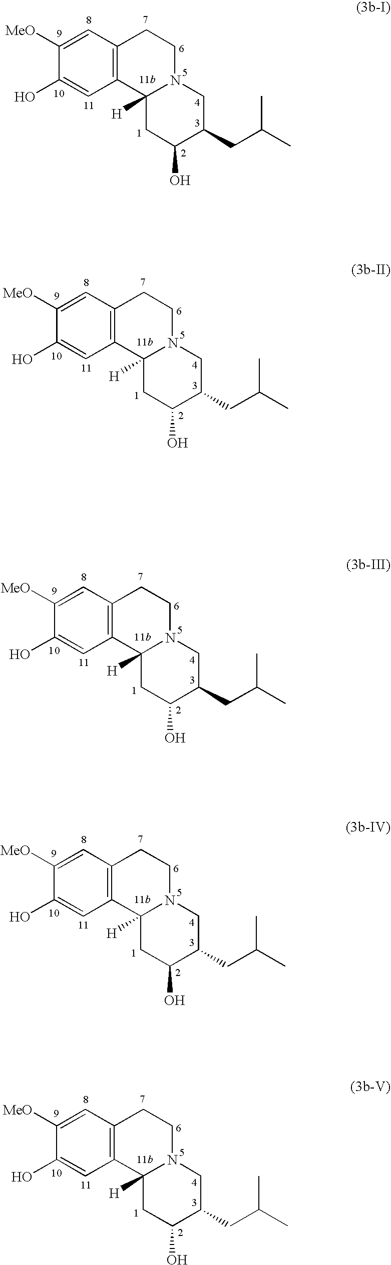 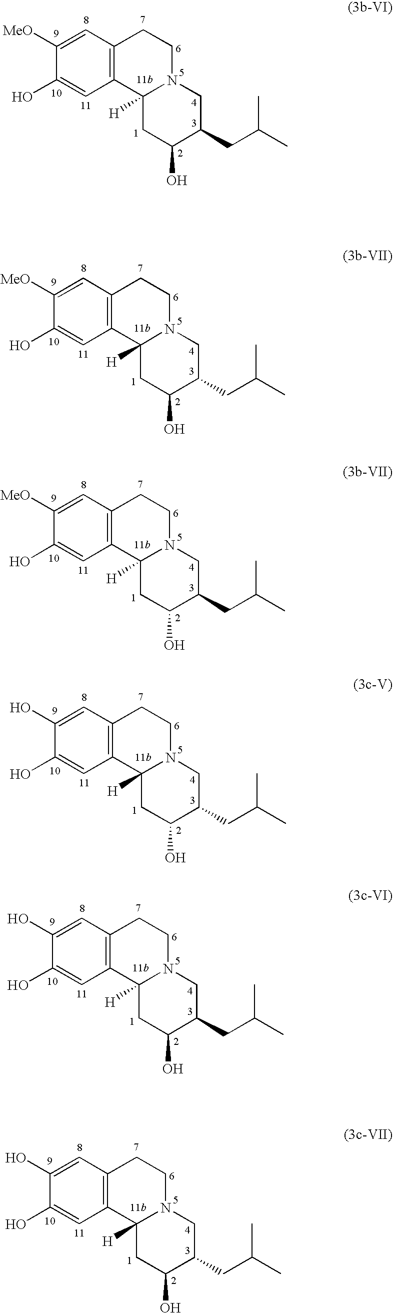 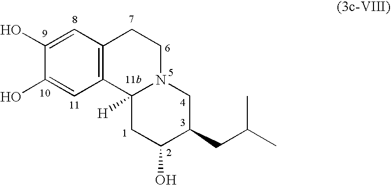
and combinations thereof, or pharmaceutically acceptable salts thereof.

27. The composition of claim 23, wherein the compound is (−)-10-desmethyl-γ-dihydrotetrabenazine.

28. A method of treating a movement disorder in a patient in need thereof comprising administrating to the patient the composition of claim 23 to thereby treat the movement disorder in the patient.

29. The method of claim 28, wherein the movement disorder is Huntington's disease.

30. The method of claim 28, further comprising halting or slowing the development of Huntington's disease.

31. The method of claim 28, wherein the movement disorder is Tourette's syndrome.

32. A method of treating an inflammatory disease in a patient in need thereof comprising administrating to the patient the composition of claim 23 to thereby treat the inflammatory disease in the patient.

33. A method of treating multiple sclerosis in a patient in need thereof comprising administrating to the patient the composition of claim 23 to thereby treat the multiple sclerosis in the patient.

34. A method of treating a psychoses in a patient in need thereof comprising administrating to the patient the composition of claim 23 to thereby treat the psychoses in the patient.

35. The method of claim 34, wherein the psychoses is schizophrenia.

36. The method of claim 35, where the method further treats a cognitive deficit associated with schizophrenia.

37. A method of treating anxiety in a patient in need thereof comprising administrating to the patient the composition of claim 23 to thereby treat the anxiety in the patient.

38. A method of treating depression in a patient in need thereof comprising administrating to the patient the composition of claim 23 to thereby treat the depression in the patient.

39. A method of treating a cognitive deficit in a patient in need thereof comprising administrating to the patient the composition of claim 23 to thereby treat the cognitive deficit in the patient.

40. A method of treating dementia in a patient in need thereof comprising administrating to the patient the composition of claim 23 to thereby treat the dementia in the patient.

41. A method of treating asthma in a patient in need thereof comprising administrating to the patient the composition of claim 23 to thereby treat the asthma in the patient.

42. A method of treating diabetes mellitus in a patient in need thereof comprising administrating to the patient the composition of claim 23 to thereby treat the diabetes mellitus in the patient.
